(19) United States
(12) Patent Application Publication
(10) Pub. No.: US 2025/0132035 A1
(43) Pub. Date: Apr. 24, 2025

(54) Non-Invasive Blood Glucose Prediction by Neural Network Based on Implicit HbA1c

(71) Applicant: Academia Sinica, Taipei (TW)

(72) Inventors: Fu-Liang Yang, Hsinchu City (TW); Justin Chu, Taoyuan City (TW)

(21) Appl. No.: 18/789,797

(22) Filed: Jul. 31, 2024

Related U.S. Application Data
(63) Continuation-in-part of application No. 17/876,579, filed on Jul. 29, 2022.

(51) Int. Cl. G16H 50/00; G06N 3/0464; G06N 3/09

(52) U.S. Cl. CPC G16H 50/00; G06N 3/0464; G06N 3/09

(57) ABSTRACT

The NIBG neural network prediction system based on implicit HbA1c of the present invention comprising a neural network configured to predict BG level of a subject based on implicit HbA1c and cardiovascular signal obtained from the subject. In an embodiment, the subject is not undergoing medical treatment that can affect cardiovascular system of the subject and the neural network is trained using training data from training cohort in which each member of the training cohort is not undergoing medical treatment that can affect cardiovascular system of the subject.

### FIELD OF THE INVENTION

The present invention is generally related to a non-invasive system and method for predicting blood glucose level of a subject using neural network based on implicit HbA1c.

Statement Regarding Prior Disclosures by the Inventor or a Joint Inventor The article Chu, J.; Chang, Y.-T.; Liaw, S.-K.; Yang, F.-L. Implicit HbA1c Achieving 87% Accuracy within 90 Days in Non-Invasive Fasting Blood Glucose Measurements Using Photoplethysmography. Bioengineering 2023, 10, 1207. https://doi.org/10.3390/bioengineering10101207 is a grace period inventor-originated disclosure under AIA 35 U.S.C. 102 (a) (1). The authors of the article Liaw and Chang are graduate students working under direction and supervision of the named inventors Fu Liang YANG and Justin Chu, and Liaw and Chang did not contribute to the conception of the claimed invention.

### BACKGROUND OF THE INVENTION

Non-invasive blood glucose (NIBG) measurement refers to the process of determining blood sugar levels without the need for traditional methods that require pricking the skin to obtain a blood sample. Traditional finger-prick measurements can cause pain and carry a risk of infection, which might discourage individuals who need to monitor their blood glucose regularly. NIBG measurement offers a more comfortable and less intrusive way to monitor glucose levels.

Numerous methods based on diverse technologies have been explored for NIBG measurement. These include enzymatic methods that test saliva, tears, and body sweat [5,6,7], electromagnetic wave sensing methods that cover a wide area of the electromagnetic spectrum [8,9,10], and transdermal methods that measure the user's bioimpedance [11].

Among these, cardiovascular signals such as photoplethysmography (PPG) stands out as a highly promising technology due to the fact that it is very easy to use and has very versatile applications [12]. PPG is a technology that monitors the light-absorption changes on the measured site. PPG sensors consist of a light-emitting diode (LED) that provides a stable light source, and light sensors that monitor the light intensity. While the blood volume at the measured site changes with pulsation, the light intensity also changes due to absorption and scattering. PPG allows unobtrusive continuous measurement while requiring only a single point of contact. Recent studies have even presented a breakthrough with contactless camera PPG for long-term, contactless, and continuous monitoring [13]. Various PPG-based applications are already being incorporated into commercially available products for the measurement of SpO2, stress levels, and blood pressure, and the detection of arrhythmias like atrial fibrillation.

Although the potential of relying on PPG or other types of cardiovascular signals alone to accurately estimate fasting blood glucose has frequently been explored via promising experimental results, a definitive answer remains elusive. We are of the opinion that the primary challenge stems from the missing variable or correction factor that addresses personal deviation. Every individual is inherently distinct, not only genetically but also in terms of their lifestyle and diet. Consequently, this gives rise to significant and undocumented variations among individuals when attempting to construct models for precise blood glucose level estimation.

Numerous NIBG studies based on PPG technology have been published over the past decades, employing a variety of different methods [14]. However, many of these studies suffer from small sample sizes and potentially compromise their generalizability. The most commonly employed PPG-extracted features are the morphological and heart rate variance features, which are correlated with an individual's vascular function and autonomic neuropathy [15,16]. Many studies also explore features in different domains, utilizing techniques such as fast Fourier transform (FFT), Kaiser-Teager energy (KTE), and spectral entropy [17]. It is worth noting that using an excessive number of features can lead to overfitting, while using too few features may result in a lack of vital information required for accurate blood glucose level estimation. Therefore, there is a need for an accurate yet simple standard for a medical NIBG.

### SUMMARY OF THE INVENTION

A non-invasive blood glucose (NIBG) prediction system comprising a cardiovascular signal input configured to input one or more cardiovascular signals of a subject into the system, a processor comprising a signal processor wherein the signal processor is configured to perform signal processing on the one or more cardiovascular signals to extract one or more cardiovascular signal features and the processor further comprising a neural network (NN) wherein the NN is configured to predict a subject's blood glucose level based on implicit HbA1c; wherein the NN comprises one or more variables; wherein the one or more variables comprises the one or more cardiovascular signal features; wherein the NN predicts blood glucose level of the subject using the one or more variables and the implicit HbA1c; and wherein the implicit HbA1c is HbA1c derived from the NN in a pretest phase based on finger prick blood glucose level of the subject and the one or more variables wherein the pretest phase occurs prior to blood glucose level prediction phase but after training phase of the NN.

A method for non-invasive blood glucose (NIBG) prediction based on implicit HbA1c comprising the steps of: training a neural network (NN) of the processor during training phase using explicit HbA1c, finger prick blood glucose level, and one or more variables obtained from each member of a training cohort so that the NN learns one or more relationships between the HbA1c, the blood glucose level, the one or more variables wherein the one or more variables comprise one or more cardiovascular signal features; inputting one or more cardiovascular signals of a subject to a processor; processing the one or more cardiovascular signals of the subject using the processor to obtain one or more cardiovascular signal features of the subject; obtaining explicit HbA1c and finger prick blood glucose level from the subject wherein explicit HbA1c is HbA1c measured from the subject using medical diagnostic devices and finger prick blood glucose level is blood glucose level measured from the subject using medical diagnostic devices; deriving an implicit HbA1c of the subject from the trained NN during pretest phase of the NN based on the finger prick blood glucose level of the subject, one or more variables of the subject and the one or more relationships between HbA1c, blood glucose level, one or more variables learned by the NN during the training phase; and predicting blood glucose level of the subject using the trained NN based on the implicit HbA1c of the subject.

### BRIEF DESCRIPTION OF THE DRAWINGS

FIG. 1 depicts a general overview of an embodiment of NIBG neural network prediction system of the present invention.

FIG. 2 depicts in detail an embodiment of the processor 200 of the NIBG neural network prediction system of the present invention.

FIGS. 3A-D depict four embodiments of the neural network (NN) of the NIBG prediction system of the present invention. FIG. 3A depicts a general structure of the NN 230 of the NIBG prediction system of the present invention. FIG. 3B depicts an embodiment of the fully connected neural network (FCNN) 301 of the present invention. FIG. 3C depicts an embodiment of the convolution neural network (CNN) 302 of the NIBG prediction system of the present invention comprising a single CNN module. FIG. 3D depicts an embodiment of the CNN 303 of the NIBG prediction system of the present invention comprising two CNN modules in parallel.

FIGS. 4A-D depicts various PPG signal of the present invention. FIG. 4A depicts an exemplary PPG signal of the present invention. FIG. 4B depicts an exemplary PPG signal of the present invention filtered by a low pass filter. FIG. 4C depicts an exemplary PPG signal of the present invention filtered by a high pass filter. FIG. 4D depicts an exemplary signal window derived from the PPG signal of the present invention facilitated by low and high pass filters.

FIG. 5A illustrates correlation of HbA1c and blood glucose level for subjects undergoing medical treatment. FIG. 5B illustrates correlation of HbA1c and blood glucose level for subjects not undergoing any medical treatment.

FIG. 6 illustrates an embodiment of the process flow of the training phase of the NIBG neural network prediction method of the present invention.

FIG. 7 illustrates an embodiment of the process flow of the pretest phase of the NIBG neural network prediction method of the present invention in which implicit HbA1c is derived.

FIG. 8 illustrates an embodiment of the process flow of the prediction phase of the NIBG neural network prediction method of the present invention.

FIG. 9 illustrates an embodiment of the process for determining implicit HbA1c. A range of HbA1c values, ranging from 4 to 12, is employed to estimate blood glucose levels (BGL). Among these values, the HbA1c value corresponding to the predicted BGL closest to the pretest reference BGL is identified and designated as the implicit HbA1c value.

FIG. 10A illustrates difference in implicit HbA1c and corresponding explicit HbA1c, and FIG. 10B illustrates distribution of the BG-explicit HbA1c relationship for the training and testing sets.

FIG. 11 illustrates CEG analysis of using implicit HbA1c and explicit HbA1c for model prediction on subjects not affected by drugs or undergoing medical treatments that can affect cardiovascular system of the subject.

FIG. 12 illustrates percentage error distribution of the prediction while using implicit and explicit HbA1c for conducting the nonparametric Wilcoxon signed-rank test. The implicit HbA1c method demonstrates a distribution pattern that resembles a normal distribution. The explicit HbA1c method exhibits a left-skewed distribution with systemically lowered prediction.

FIGS. 13A-E illustrate an embodiment of detailed neural network (NN) structure of the present invention. The NN uses two input layers for signal vector and feature vector. The signal vector takes in the segmented PPG waveform data while feature vector takes in the HbA1c value and other PPG extracted features.

### DETAILED DESCRIPTION OF THE INVENTION

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof. “About” in the context of amount values refers to an average deviation of maximum ±20%, ±10% or ±5% based on the indicated value. For example, an amount of about 30% refers to 30% ±6%, 30% ±3% or 30% ±1.5%.

A “subject,” “individual” or “patient” is used interchangeably herein, which refers to a vertebrate, preferably a mammal, more preferably a human.

“Neural network,” a subset of Artificial Intelligence (AI), comprises a network of nodes each capable of communication with one or more other nodes wherein each nodes of the network acts as a neuron able to learn and mimic the human thinking process. The neural network typically comprises an input layer, one or more hidden layers and an output layer. Various neural network frameworks may comprise but not limited to Caffee, Keras, Microsoft Cognitive Toolkit, MXNET, DeepLearning4j, Chainer and TensorFlow. Deep learning neural network is a subset of neural network comprising three or more hidden layers.

The term “photoplethysmography (PPG)” is a technology that monitors the light-absorption changes on the measured site caused by biological processes such as blood volume changes in the microvascular bed of tissue. While the blood volume at the measured site changes with pulsation, the light intensity also changes due to absorption and scattering. PPG allows unobtrusive continuous measurement while requiring only a single point of contact. Recent studies have even presented a breakthrough with contactless camera PPG for long-term, contactless, and continuous monitoring [13]. Various PPG-based applications are already being incorporated into commercially available products for the measurement of SpO2, stress levels, and blood pressure, and the detection of arrhythmias like atrial fibrillation. A PPG is often obtained by using a pulse oximeter which illuminates the skin and measures changes in light absorption. [3] A conventional pulse oximeter monitors the perfusion of blood to the dermis and subcutaneous tissue of the skin. An example of a PPG sensor may consist of a light-emitting diode (LED) that provides a stable light source, and light sensors that monitor the light intensity.

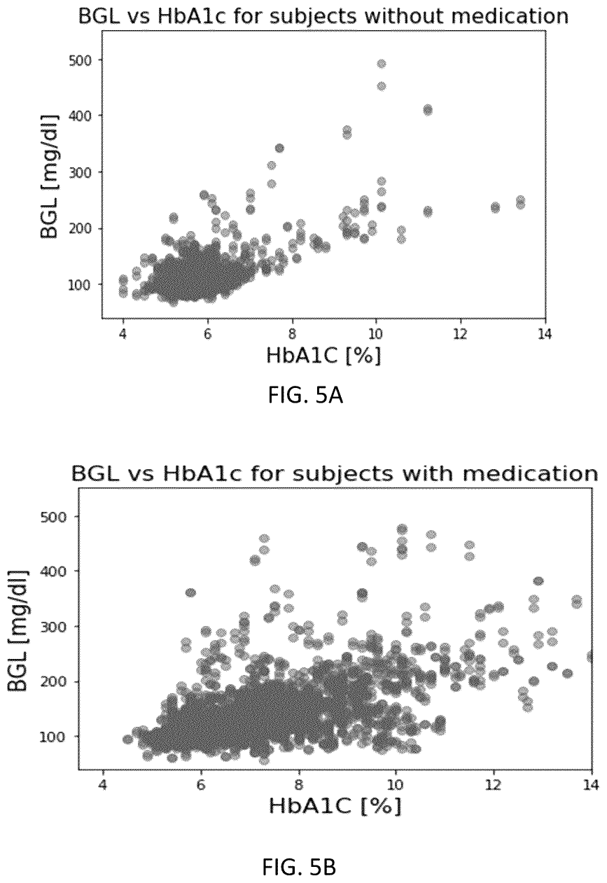

“HbA1c” is the hemoglobin in the bloodstream that becomes chemically bonded with glucose over time. It has been recognized as one of the powerhouse indexes on diagnosing diabetes and identifying prediabetes by the American Diabetes Association. Measuring HbA1c level can help one to determine their average BG level and can be conducted at any time without prior dietary preparation (e.g., fasting). Unlike blood glucose tests, HbA1c is not affected by day-to-day variability. HbA1c value can be represented in either Diabetes Control and Complication Trial units (DCCT, %), or International Federation of Clinical Chemistry units (IFCC, mmol/mol). For the present disclosure, all HbA1c values are reported in the DCCT units with percentage (%). The measured HbA1c reflects the average blood glucose level of approximately the past three months due to the limited lifespan of red blood cells. The recommended cut-off value of HbA1c for diagnosing diabetes is at 6.5%. But HbA1c value less than 6.5% does not rule out the possibility of diabetes [24]. Although there exists a correlation between HbA1c and BG level, they are not directly interchangeable, as shown in FIGS. 5A and 5B.

The phrase “medical treatment” includes any medical procedures, drug or drug regimen regulated by the United States Food and Drug Administration and/or European Medicines Agency as a medical procedure, drug or drug regimen. The present invention provides a non-invasive blood glucose (NIBG) neural network prediction system that predicts blood glucose using implicit HbA1c. Implicit HbA1c is distinct from measured HbA1c which herein is referred to as explicit HbA1c. Specifically, measured or explicit HbA1c is the HbA1c value measured directly from a subject such as HbA1c measured from a subject using known HbA1c measurement devices and methods used in hospitals and specialized clinics. In contrast, in an embodiment, the implicit HbA1c is HbA1c calculated or derived from blood glucose predicting machine learning systems or methods configured to relate HbA1c, blood glucose levels and one or more variables such as features extracted from personal physiological data and/or cardiovascular signals such as PPG and/or ECG signals and/or A-line pressure transducer signals etc. Specifically, in an embodiment, since blood glucose predicting machine learning methods and systems such as the system of the present invention comprise relationships between HbA1c, blood glucose level and the one or more variables, it is possible to then use such methods and systems to derive implicit HbA1c based on blood glucose level, the one or more variables and the relationships between HbA1c, blood glucose level and the one or more variables. In other words, implicit HbA1c reflects the HbA1c value that the machine learning system or method anticipates given a specific blood glucose level and the one or more variables. In an embodiment, this implicit HbA1c value may then be used for blood glucose predicting using the machine learning system of the present invention for the next two to three months.

Importantly, in an embedment, the relationships between the blood glucose level, HbA1c and the one or more variables capture personal deviations that are learned during training phase of the blood glucose level predicting machine learning system and method. Therefore, the implicit HbA1c value derived from these relationships, blood glucose level and the one or more variables also encompasses systematic correction elements that account for personal deviations, enhancing the accuracy of blood glucose level predictions. In an embodiment, these personal deviations may comprise medication or medical procedures of the subject, biological differences between the subjects, lifestyle variations of the subjects etc.

This idea can be expressed in terms of functions. For example, a system and method comprising machine learning model that predicts BGL can generally be represented as Equation (1). Here, the function ML( ) symbolizes the machine learning model, while F1 through Fn correspond to the diverse set of features that collectively contribute to achieving an accurate prediction of the blood glucose level.

$$BGL = ML ( F 1 , F 2 , F 3 … Fn ) \qquad ( 1 )$$

While different methods may employ different features, our prior work demonstrated that BGL can be accurately estimated by a machine learning model with cardiovascular signal (CS) and HbA1c input, albeit under certain conditions. This leads us to modify Equation (1) into Equation (2a):

$$BGL = ML ( CS , HbA 1 c ) \qquad ( 2 a )$$

However, it is important to acknowledge the intricate interplay of personal deviations such as medication, individual differences, lifestyle variations, and more, which may not have been fully accounted for. This realization prompts us to introduce the correction item ΣCi into the equation. For subjects not undergoing treatment with drugs, the effects of ΣCi may not be significant enough to seriously hinder the prediction performance, but it is undeniable that these effects still exist. Consequently, we further revise the equation into Equation (2b).

$$BGL = ML ( CS , HbA 1 c , \sum Ci ) \qquad ( 2 b )$$

These personal difference effects were dealt with by using a personalized deduction learning model that required multiple measurements from the user in our previous work [22]. Other works sought to account for these deviations by utilizing numerous personal profiles [23]. Without being bound to theory, the system and method of the present invention leverage the concept of implicit HbA1c to achieve a similar effect.

The implicit HbA1c is determined by substituting HbA1c and BLG in Equation (2). It is like solving a multi-variate polynomial function with only one unknown variable. To solve for the unknown HbA1c value, the model is provided with a range of HbA1c inputs, generating a series of predictions. By cross-reference these predictions with the known BGL value, we can determine which corresponding HbA1c produces the most accurate estimation. This process not only yielded an HbA1c estimate, but it also accounted for the aforementioned correction items ΣCi. In other words, implicit HbA1c is the HbA1c value that has been adjusted to accommodate an individual's specific correction items or personal deviations as referred to hereinafter. Thus, this refinement further transforms Equation (2b) into Equation (3)

$$BGL = ML ( CS , HbA 1 {c}_{imp} ) \qquad ( 3 )$$

wherein HbA1cᵢₘₚ is implied HbA1c

HbA1c reflects an average BGL, and its correlation with fasting BGL is influenced by individual lifestyle, such as constant high BGL during the day and multiple meals. Consequently, the relationship between each individual's HbA1c and fasting blood glucose follows a unique curve. For instance, individuals with prediabetes may still have a pancreas capable of producing a sufficient amount of insulin to maintain normal fasting BGL, but their daily BGL may fluctuate in a big range depending on diet and lifestyle. The system and method of the present invention should be effective in predicting BGL across various demographics, including different races, ages, and genders, as it effectively compensates for personal deviations arising from miscellaneous correction factors using implicit HbA1c.

Evidence of the improvement by the use of implicit HbA1c can be seen in the Examples below in which accuracy of BGL prediction is improved by about 16% as compared to prediction using the same system and method but using explicit HbA1c rather than implicit HbA1c. Without being bound to theory, the increased accuracy may be attributed to the ability of blood glucose predicting machine learning system and method that uses implicit HbA1c to better capture the personal deviations than the use of explicit HbA1c as discussed above.

A further advantage of using implicit HbA1c rather than explicit HbA1c is that, as well known in the art, devices and equipment required to obtain HbA1c data are usually only available in hospitals and specialized clinics in contrast to devices needed to obtain cardiovascular signal such as PPG and blood glucose data which are easily accessible at a subject's home. The ability to acquire a simple alternative HbA1c vastly improves the usability of prediction models that utilize HbA1c.

To further improve the accuracy of the system and method of the present invention, an embodiment of the system and method of the present invention eliminates a major personal deviation which is a subject's medical treatment as discussed above which is shown to have significant negative effect on NIBG prediction accuracy. Specifically, since cardiovascular signal based NIBG prediction relies on the correlation of BG level with cardiovascular signal obtained from the subject as a representation of the subject's cardiovascular system, medical treatment on cardiovascular disease may influence the cardiovascular system and, therefore, the cardiovascular signal such that its correlation to blood glucose (BG) level is substantially altered or even eliminated.

Therefore, in an embodiment the NIBG neural network prediction system of the present invention comprises a neural network configured to predict BG level of a subject wherein the subject is not undergoing medical treatment that can affect the subject's cardiovascular system and the neural network is trained using training data from each member of a training cohort in which each member is not undergoing medical treatment that can affect cardiovascular system wherein the training data is based upon cardiovascular signal, explicit HbA1c and reference BGL obtained from each member of the training cohort. In another embodiment, the NIBG neural network prediction system of the present invention predicts BG level of a subject based on implicit HbA1c wherein the subject is not undergoing medical treatment that can affect the subject's cardiovascular system and the neural network is trained using training data from training cohort in which each member is not undergoing medical treatment that can affect the cardiovascular system wherein the training data is based upon cardiovascular signal, explicit HbA1c and reference BG obtained from each member of the training cohort.

In an embodiment, medical treatments that can affect cardiovascular system comprises high blood pressure and high blood pressure prevention related drugs and treatments, stroke and stroke prevention drugs and treatments, and/or heart attack and heart attack prevention drugs and treatments, or a combination thereof. In an embodiment, medical treatments that can affect cardiovascular system comprises high blood pressure and high blood pressure prevention related drugs and treatments, stroke and stroke prevention drugs and treatments, and/or heart attack and heart attack prevention drugs and treatments or a combination thereof that are regulated by the United States FDA and or European Medicine Agency. In an embodiment, medical treatments that can affect cardiovascular system consists of high blood pressure and high blood pressure prevention related drugs and treatments, stroke and stroke prevention drugs and treatments, and/or heart attack and heart attack prevention drugs and treatments or a combination thereof. In an embodiment, medical treatments that can affect cardiovascular system consists of high blood pressure and high blood pressure prevention related drugs and treatments, stroke and stroke prevention drugs, and/or treatments and heart attack and heart attack prevention drugs or a combination thereof that are regulated by the United States FDA and or European Medicine Agency.

Moreover, any medical treatments not directly affecting the cardiovascular system such as diabetes drugs or treatments such as insulin injections can also be used as a criteria to exclude a subject and training cohort to improve accuracy of BG prediction. Therefore, in an embodiment, medical treatments used to exclude subject and training cohort for the present invention comprise medical treatments that can affect cardiovascular system as defined above as well as medical treatment related to diabetes that are regulated by the United States FDA and or European Medicine Agency such as insulin injections.

Furthermore, in an embodiment, to more precisely refine or define the method and system of the present invention's prediction accuracy, subjects or training cohort undergoing any medical treatments are excluded regardless of whether the medical treatment affects cardiovascular system or is used for treating diabetes. Such medical treatment includes any medical treatment, drug or drug regimen recognized as medical treatment, drug or drug regimen by the United States Food and Drug Administration and/or the European Medicines Agency regardless of whether the medical treatment affects cardiovascular system or are for treating diabetes. Therefore, in an embodiment the NIBG neural network prediction system of the present invention comprises a neural network configured to predict BG level of a subject wherein the subject is not undergoing any medical treatment and the neural network is trained using training data from cohort not undergoing any medical treatment wherein the training data is based upon cardiovascular signal obtained from the training cohort. In another embodiment, the NIBG neural network prediction system of the present invention predicts BG level of a subject based on implicit HbA1c as well as cardiovascular signal obtained from the subject wherein the subject is not undergoing any medical treatment and the neural network is trained using training data from cohort not undergoing any medical treatment wherein the training data is based upon cardiovascular signal, explicit HbA1c and reference BG obtained from the training cohort. In an embodiment, cardiovascular signal comprises, PPG signal, ECG signal or A-line pressure transducer signals.

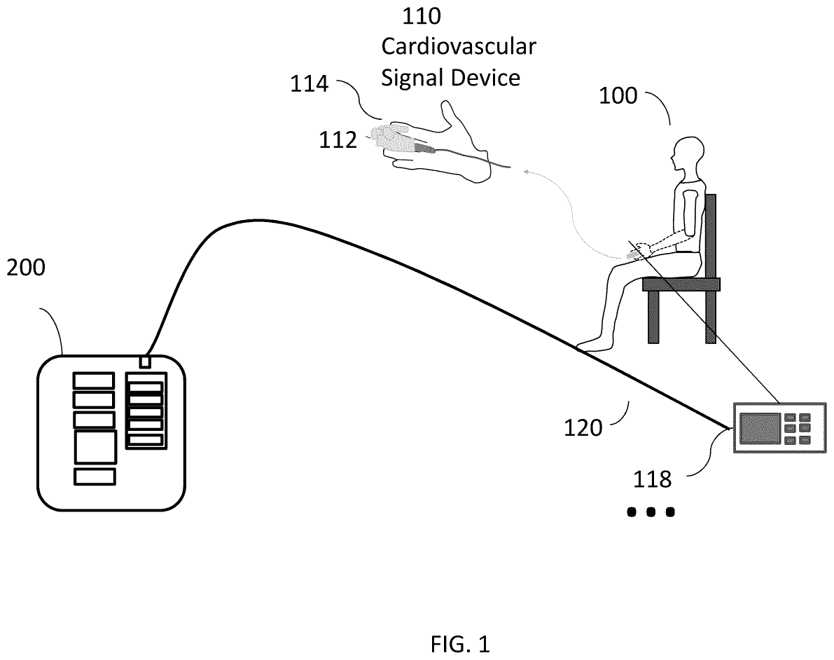

FIG. 1 illustrates an embodiment of the NIBG neural network prediction system of the present invention. As shown in FIG. 1, the system of the present invention comprises subject 100, cardiovascular signal input 110, connector 120 and processor 200. In an embodiment, the subject 100 comprises a person whose blood glucose level is to be predicted using the system and method of the present invention and may or may not be undergoing any medical treatment that can affect his or her cardiovascular system and/or is not undergoing medical treatment for diabetes. In an embodiment, the subject 100 comprises a person whose blood glucose level is to be predicted using the system and method of the present invention and is not undergoing any medical treatment that can affect his or her cardiovascular system and/or is not undergoing medical treatment for diabetes. In an embodiment, the subject 100 comprises a person whose blood glucose level is to be predicted using the system and method of the present invention and is not undergoing any medical treatment.

In an embodiment, the cardiovascular signal input 110 comprises a cardiovascular signal input such as cardiovascular signal reader 112, signal emitter 114 and a signal module 118. The cardiovascular signal reader 112 is configured to read signals emanating from the subject 100. The cardiovascular signal input 110 may further comprise a signal emitter 114 for outputting signals that pass through the body of the subject 100, then emanate out from the subject 100 to be read by the signal reader 112. In an embodiment, the cardiovascular signal reader 112 comprises a PPG signal reader, ECG signal reader, an A-line pressure transducer signal reader or a combination thereof.

The cardiovascular signal input 110 further comprises a signal module 118 configured to communicate with and control the signal reader 112 and signal emitter 114. The signal module 118 may also comprise one or more control panels that allow a user to control incoming and outgoing signals such as triggering signals and/or capturing signals.

Connector 120 is configured to allow communication between the cardiovascular signal input 110 and the processor 200. In an embodiment, the connector 120 may transmit the signal read by the cardiovascular signal input 110 to the processor 200 as well as transmit commands from the processor 200 to the cardiovascular signal input 110 to command the signal module 118 to trigger and/or read signals. In one embodiment, the connector 120 may be a physical wire. In another embodiment, the connector 120 may be a wireless connection such as those using Wi-Fi or Bluetooth technology.

In an embodiment, cardiovascular signal may have already been previously recorded and stored on a storage device capable of storing data such as a flash drive, a hard drive, a cloud etc. In such a setup, a cardiovascular signal reader 112 and signal emitter 114 are not required and the cardiovascular signal input 110 may comprise any storage device capable of storing cardiovascular signals know in the art such as a flash drive, a hard drive, a cloud etc. Such cardiovascular signal input 110 is then connected to the connector 120 that is capable of transmitting the cardiovascular signal stored on the cardiovascular signal input 110 to the processor 200.

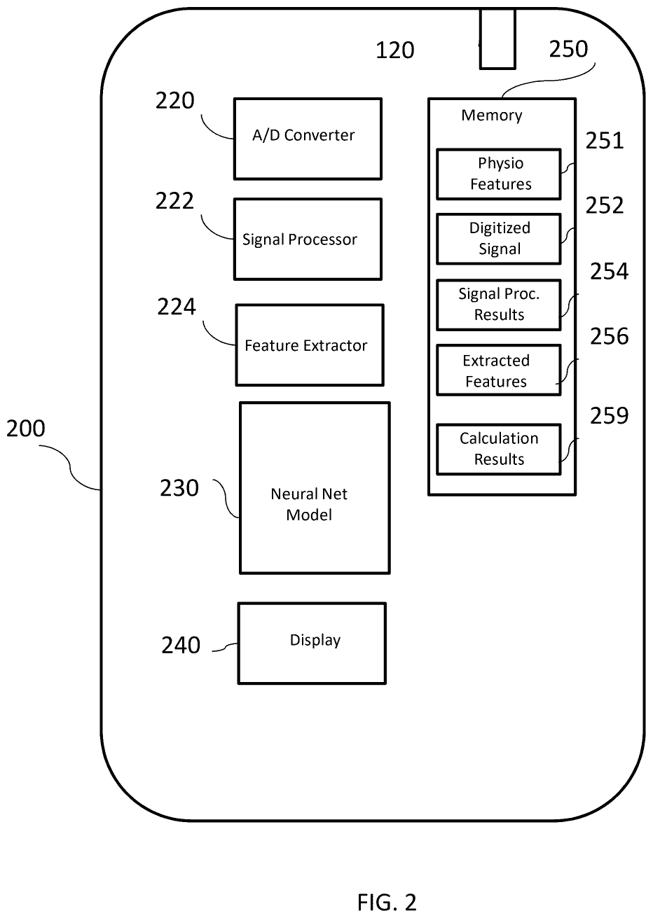

FIG. 2 illustrates processor 200 in further detail. As shown in FIG. 2, processor 200 comprises an analogue to digital converter (A/D converter) 220, signal processor 222, feature extractor 224, neural network 230, display 240 and memory 250.

It should be noted that components of the processor 200 described may reside in one single device as illustrated in FIG. 2 or reside in separate devices or in a cloud. For example, the analogue to digital converter 220, the signal processor 222, the feature extractor 224, the neural network 230 and/or memory 250 may each or in various combinations be a stand-alone device or part of a cloud. Furthermore, display 240 which is configured to allow user input into processor 200 such as via onscreen input as well as display information such as physiological measurements and/or HbA1C may be a stand-alone device.

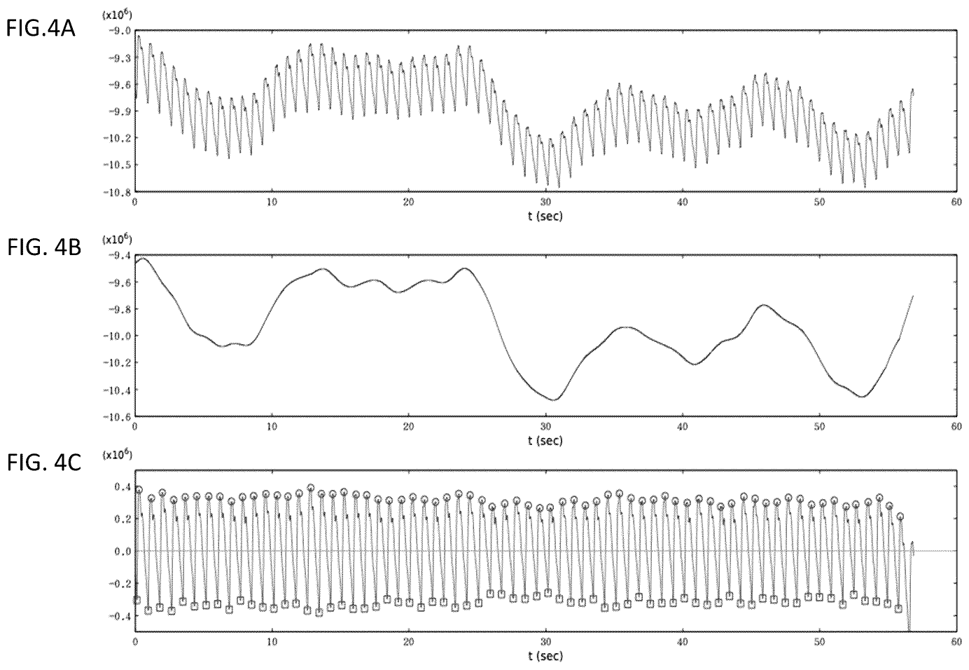

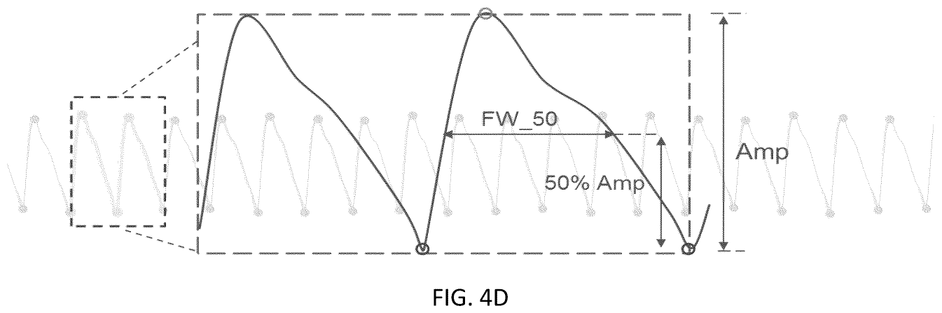

The A/D converter 220 is configured to digitize the analogue signal transmitted to the processor 200 into digitized signal 252 which may be stored in memory 250. The signal processor 222 is configured to process the digitized signal 252 to facilitate extraction of features from the cardiovascular signal. For example, in an embodiment, the signal processor 222 may be configured to decompose the signal into AC and DC components, perform Fourier transformation, etc. in order to facilitate analysis and further processing of cardiovascular signals such as digitization of the cardiovascular signals into signal windows and extraction of features 256 from the digitized signal 252 as described in further detail below in connection with FIG. 4. The signal processing results 254 may be stored in memory 250. In an embodiment, cardiovascular signal comprises PPG signal.

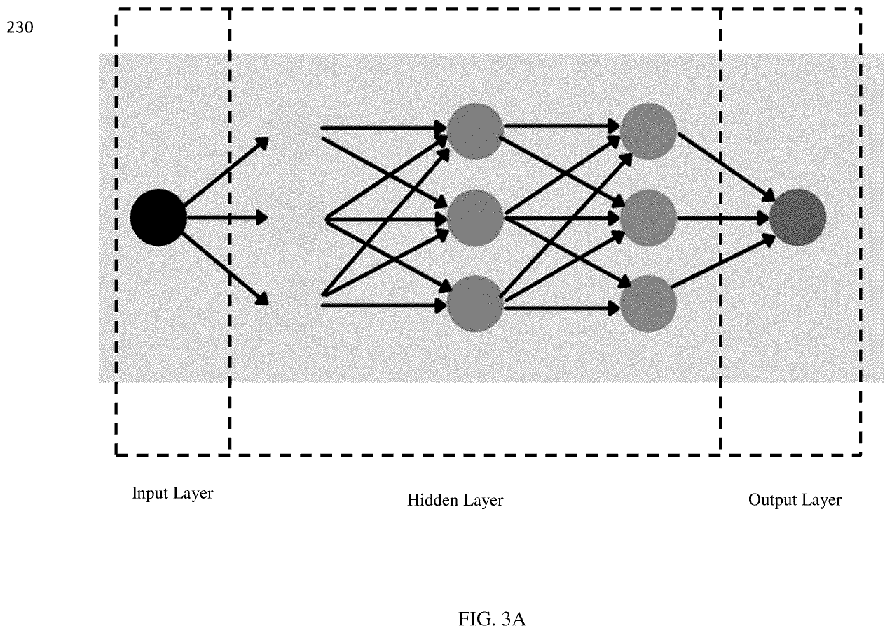

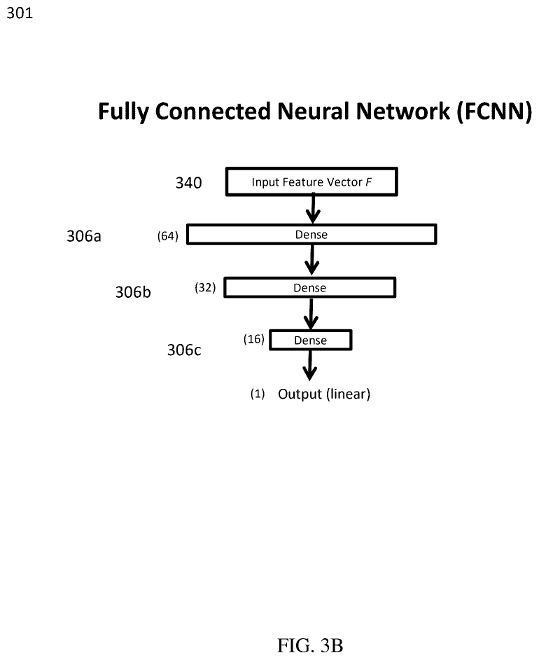

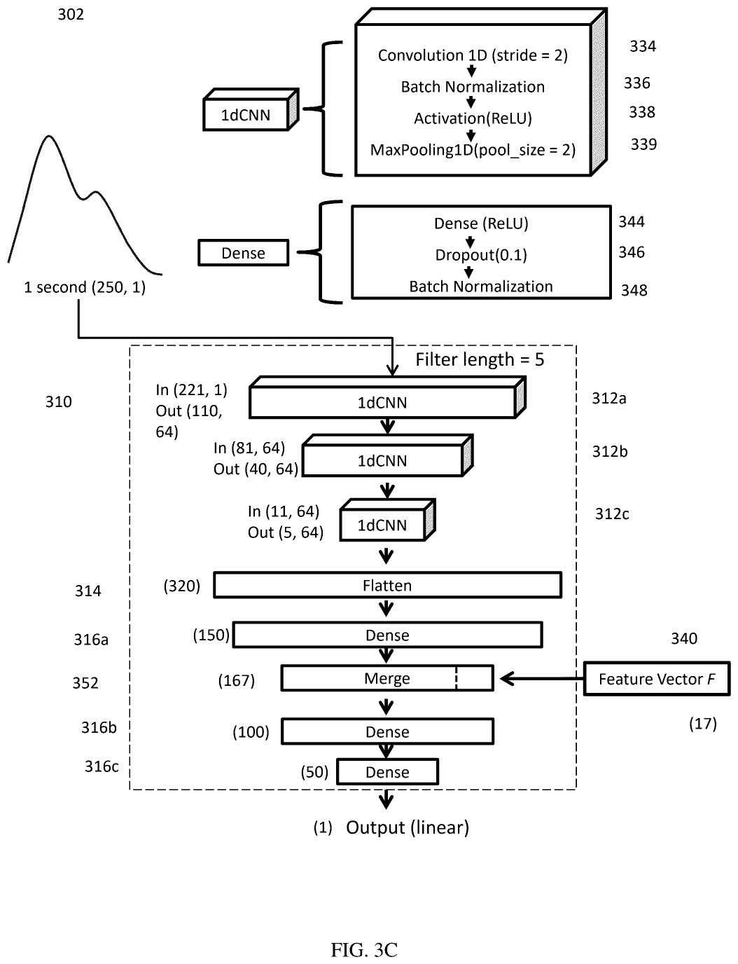

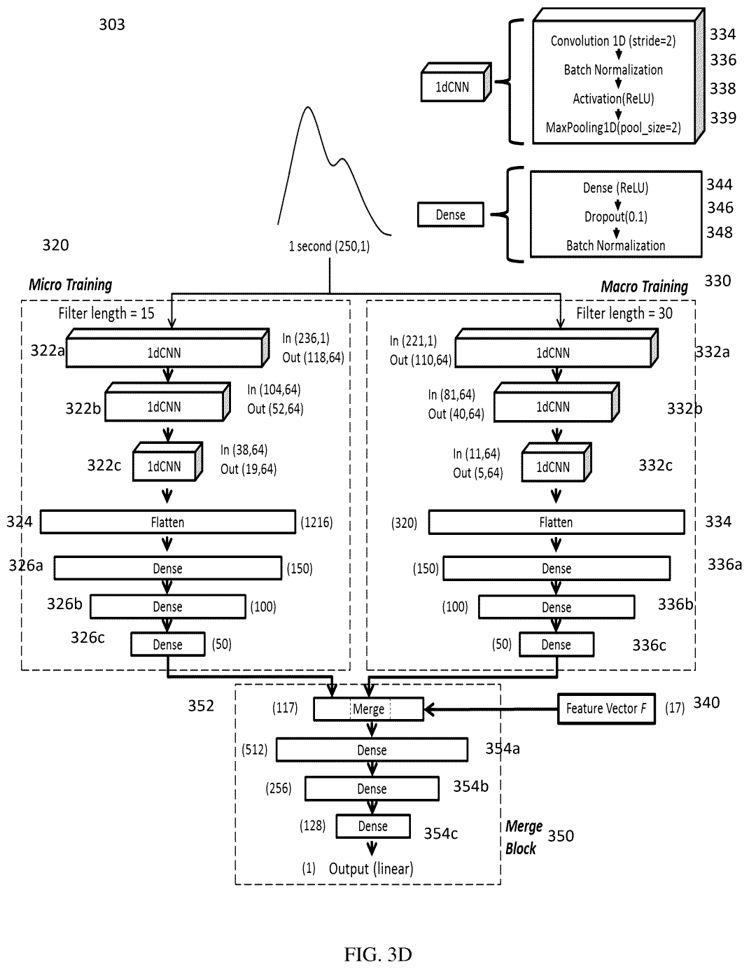

FIGS. 3A-D illustrates various embodiments of the neural network 230 of the present invention. As shown in FIG. 3A, an embodiment of the neural network 230 of the present invention comprises an input layer, one or more hidden layers and an output layer. In an embodiment, the input layer is configured to receive one or more inputs such as one or more input vectors or matrices. In an embodiment, each hidden layer comprises one or more nodes wherein each node has an associated weight and bias that may be adjusted during training phase of the model to optimize prediction accuracy. In an embodiment, each node connects to one or more nodes of a different hidden layer. In an embodiment, if the output of any individual node is above the specified threshold value, that node is activated, sending data to the next layer of the network; otherwise, no data is passed along to the next layer of the network. In an embodiment, the neural network of the present invention comprises any number of hidden layers. In an embodiment, the neural network of the present invention comprises 1 to 10 hidden layers such as 1 layer, 2 layers, 3 layers, 4 layers, 5 layers, 6 layers, 7 layers, 8 layers, 9 layers or 10 layers including all ranges and numbers falling within these values.

In an embodiment, the input of the NN 230 of the present invention may comprise one or more vectors or matrices that, in turn, comprises signal window of the cardiovascular signal, implicit or explicit HbA1c, various features or a combination thereof. In an embodiment, the input to the NN 230 of the present invention comprises one or more 1 d vectors that, in turn, comprises signal window of the cardiovascular signal, implicit or explicit HbA1c, various features or a combination thereof. The features are discussed in further details below.

In an embodiment, the NN 230 of the present invention is trained using training data such as cardiovascular signal and explicit Hba1c and/or reference BG level obtained using convention finger prick method from training cohort not undergoing any medical treatment that can affect his or her cardiovascular system as recognized as having such an effect on the cardiovascular system by the United States Food and Drug Administration and/or European Medicines Agency and/or not undergoing any medical treatment for treating diabetes. In an embodiment, the NN 230 of the present invention is trained using training data such as cardiovascular signal and explicit HbA1c and/or reference BG level obtained using convention finger prick method from training cohort not undergoing any medical treatment. In an embodiment, training of the NN 230 of the present invention comprises minimizing loss as calculated by the total of differences between BGs predicted by the system of the present invention and the corresponding reference BGs. Various neural network frameworks for building NN 230 of the present invention comprises Caffee, Keras, Microsoft Cognitive Toolkit, MXNET, DeepLearning4j, Chainer and TensorFlow.

In an embodiment, the neural network 230 of the present invention may comprise a fully connected neural network (FCNN) 301. An embodiment of the fully connected neural network (FCNN) of the present invention is illustrated in FIG. 3B. As shown in FIG. 3B, an embodiment of the FCNN of the present invention comprises an input layer, a hidden layer and an output layer. In an embodiment, the input layer is configured to receive one or more inputs such as one or more vectors or matrices. In an embodiment, the input layer is configured to receive as input personal physiological features, pulse morphological features, heart rate variance features, HbA1c or a combination thereof. In an embodiment shown in FIG. 3B, the input layer is configured to receive as input feature vector F 340 which comprises the 16 features and explicit or implicit HbA1c. As shown in FIG. 3B, the FCNN of the present invention comprises one or more hidden layers in which each hidden layer comprises a dense layer 306. In an embodiment, the dense layer 306 comprises one or more nodes in which each node is connected to each node of the preceding layer. In an embodiment, nodes of the dense layer perform operations that reduces dimension of the input to help identify important features for predicting BG. In an embodiment, nodes of the dense layer perform matrix-vector multiplication wherein the values of the matrix are trainable and maybe updated by back propagation. As shown in FIG. 3B, the FCNN of the present invention further comprises an output layer configured to output BG prediction.

In an embodiment, the neural network 230 of the present invention may comprise a convolution neural network (CNN) 302. An embodiment of the CNN 302 of the present invention is illustrated in FIG. 3C. As shown in FIG. 3C, in an embodiment, the hidden layer of the CNN 302 of the present invention comprises convolution module 310. The convolution module 310 comprises one or more convolution layers 312 such as 312a, 312b and 312c as shown in FIG. 3C. In an embodiment, the convolution module 310 may comprise any number of convolution layers. In an embodiment, the convolution module 310 may comprise 1 to 10 convolution layers.

In an embodiment, each convolution layer 312 comprises a convolution submodule 334, batch normalization module 336, activation function module 338, pooling module 339 or a combination thereof. In an embodiment, the activation module 338 is configured to perform ReLU operation. In an embodiment, the pooling module 339 is configured to perform maxpooling or average pooling operation. In an embodiment, the convolution submodule 334 is configured to perform convolution operation using one or more filters on the one or more inputs or output from another convolution layer. In an embodiment, the filter may have any length. In an embodiment, the filter may have lengths of 1 to 500 such as 1, 5, 10, 15,20, 30, 40, 60, 80, 100, 200, 250, 300, 350, 400, 450 or 500 including all ranges and numbers falling within these values.

In an embodiment, the input to the CNN 302 of the present invention may comprise one or more vectors or matrices that, in turn, comprises signal window derived from the PPG signal obtained from the subject, implicit or explicit HbA1c, various features or a combination thereof as disclosed above. In an embodiment, the input to the CNN 302 of the present invention comprises a 1 d vector that, in turn, comprises signal window derived from the PPG signal, implicit or explicit HbA1c, various features or a combination thereof as disclosed above. The output of the convolution sub module 334 may be further processed by the batch normalization module 336, activation function 338 and/or pooling module 339.

The convolution module 310 may further comprise a flatten module 314 and one or more dense layers 316. In an embodiment, the flatten module 314 is configured to reduce the dimension of the output of the convolution layers 312. Each convolution module 310 further comprises one or more dense layers 316 configured to further identify important parts of the output from flatten layer 314. In an embodiment, each dense layer 316 comprises a dense activation module 344, a dropout module 346 and/or a batch normalization module 348. In an embodiment, a second input comprising implicit or explicit HbA1c as well as various features may be separately input in the merge layer 352 configured to accept and process output from dense layer 316a and feature vector 340.

FIG. 3D illustrates another embodiment of the neural network 230 of the present invention comprising CNN 303 with two convolution modules. As shown in FIG. 3D, in an embodiment, the hidden layer of the CNN 303 of the present invention comprises two convolution modules 320 and 330 in parallel wherein each convolution module has filter of different lengths. A smaller filter length is configured to capture more detailed level aspects of the input vector whereas a larger filter length is configured to capture higher level aspects of the input vector. Therefore, in an embodiment, the convolution module with shorter length filter may be referred to as micro convolution module 320 whereas the convolution module with longer length filter may be referred to as macro convolution module 330.

In an embodiment the structure of each convolution module 320, 330 is the same as convolution module 310 but the length of filter for the micro convolution module or the micro filter 321 is shorter than that of the macro filter 331. In an embodiment, the length of micro filter 331 is about ¼ to ¾ of the macro filter 31 such as ¼, ½ or ¾ including any fractions and fraction ranges falling within these values. In an embodiment, the macro filter may have any length. In an embodiment, the macro filter may have lengths of 1 to 500 such as 1, 5, 10, 15, 20, 30, 40, 60, 80, 100, 200, 250, 300, 350, 400, 450 or 500 including all lengths and length ranges falling within these values.

In an embodiment, the micro convolution module 320 comprises one or more micro convolution layers 322 such as 322a, 322b and 322c as shown in FIG. 3D. The macro convolution module 330 comprises one or more macro convolution layers 332 such as 332a, 332b and 332c as shown in FIG. 3D. In an embodiment, each convolution module 320, 330 may comprise 1 to 10 convolution layers.

Each convolution module 320, 330 may further comprise a flatten module 324, 334, one or more Dense layers 326, 336 or a combination thereof. In an embodiment, the flatten module 324, 334 is configured to reduce the dimension of the output of the convolution layers 322, 332. In an embodiment, each convolution module 320, 330 may further comprise one or more dense layers 326, 336 configured to further identify important parts of the output from flatten layer 324, 334. In an embodiment, each dense layer 326,336 comprises a dense activation module 344, a dropout module 346, a batch normalization module 348, or a combination thereof.

In an embodiment, the convolution sub module 334 is configured to perform convolution operation using one or more filters on the one or more inputs or output from another convolution layer.

In an embodiment, the CNN 303 of the present invention further comprises a merge block module 350 configured to receive feature vector or matrix 340 as well as results of the micro and macro convolution modules 320, 330 in order to analyze and output a predicted BG level of the subject based on these inputs. In an embodiment, the merge block module 350 comprises a merge layer 352 that merges outputs from the two CNN modules and feature vector 340 for further processing. In an embodiment, the merge block module 350 further comprises one or more dense layers 354 configured to process the merged inputs to output a predicted BG level for the subject. In an embodiment, the feature vector or matrix 340 may comprise various features disclosed herein, including personal physiological features, pulse morphological features, heart rate variance features or a combination thereof.

The feature extractor 224 is configured to extract features from the digitized signal 252 and/or results of the signal processing 254. In an embodiment, extracted features 256 may comprise any features disclosed herein and may be used to create the feature vector 340 as input to the various embodiments of the NN 230 of the present invention.

In an embodiment, input for any embodiments of the NN 230 of the present invention may comprise several input types such as digitized segment of the cardiovascular signal obtained from the subject, explicit or explicit HbA1c, various features or a combination thereof. In an embodiment, the various input types for the NN 230 of the present invention may comprise one or more input vectors or matrices. For example, the digitized segment of the cardiovascular signal may comprise one input vector or matrix while the features comprise a different input vector or matrix such as the feature vector 340. Alternatively, one or more input types may comprise one single input vector or matrix such that the digitized segment of the cardiovascular signal and the features are concatenated to form one input vector or matrix. In an embodiment, each input vector or matrix comprises a one dimensional (1d) input vector. In another embodiment, the cardiovascular signal comprises an input vector whereas the features including implicit or explicit HbA1c comprises a separate vector such as the features vector 340 wherein the cardiovascular signal is directly input to the convolution modules and the features are input separately such as in the merge layer 352. In an embodiment, the cardiovascular signal comprises PPG, ECG or A-line pressure transducer signals.

FIG. 4A illustrates an exemplary cardiovascular signal in the form of a PPG signal. In an embodiment, the digitized segments of the PPG signal comprise signal windows as shown in FIG. 4D. In an embodiment, each signal window comprises or consists of a fixed time length of the PPG signal of about 1 second to about 20 seconds such as about 1 second, about 1.6 seconds about 2 seconds about 5 seconds about 10 seconds, about 15 second or about 20 seconds. In an embodiment, each signal window starts from each valley of the PPG signal such that, if a subject's heart beats 60 beats/second, then a 1-minute-long PPG signal would result in 60 signal windows. In an embodiment, the signal windows may be better defined using a frequency filter as illustrated in the FIGS. 4B and 4C to better identify peaks and troughs of the PPG signal as shown in FIG. 4D. In an embodiment, the valleys of

the PPG waveform were identified using the Bigger-Fall-Side algorithm [26]. Then from each valley, a backwardly one-second-long segment (total of 250 data points) containing the pulse was extracted. Averaging over the pulses, it was used to represent the entire minute of the PPG signal for deep learning neural network. An example of a signal window of the present invention is illustrated in FIG. 4D.

In an embodiment, features may comprise personal physiological features, pulse morphological features, heart rate variance features or a combination thereof. In an embodiment, the pulse morphological features and heart rate variance features may also be termed extracted features 256 since these comprise features derived from the cardiovascular signals. In an embodiment, the personal physiological features 251 may comprise age, waist circumference, body mass index, systolic, diastolic blood pressure or a combination thereof. In an embodiment, the pulse morphological features derived from the averaged cardiovascular signal pulse may comprise the width of the pulse at 50% height, total pulse area of the minute, average pulse area, the median of the pulse area, or time difference from pulse valley to peak or a combination thereof. In an embodiment, the heart rate variance features derived from the cardiovascular signal window may comprise both low-frequency power from Fast Fourier Transformation (FFT), high-frequency power from FFT, total power from FFT, percentage of pulse successive interval changes exceeding 20 ms, the standard deviation of successive interval changes or a combination thereof. In an embodiment, all 17 features listed above (plus HbA1c for models including it) is aggregated into a feature vector F 340 as input into the neural network of the present invention. In another embodiment, other features such as peak and dicrotic notch location, peak amplitude, peaks and valleys location on 1st and 2nd derivative of the signal waveform may also be included as features

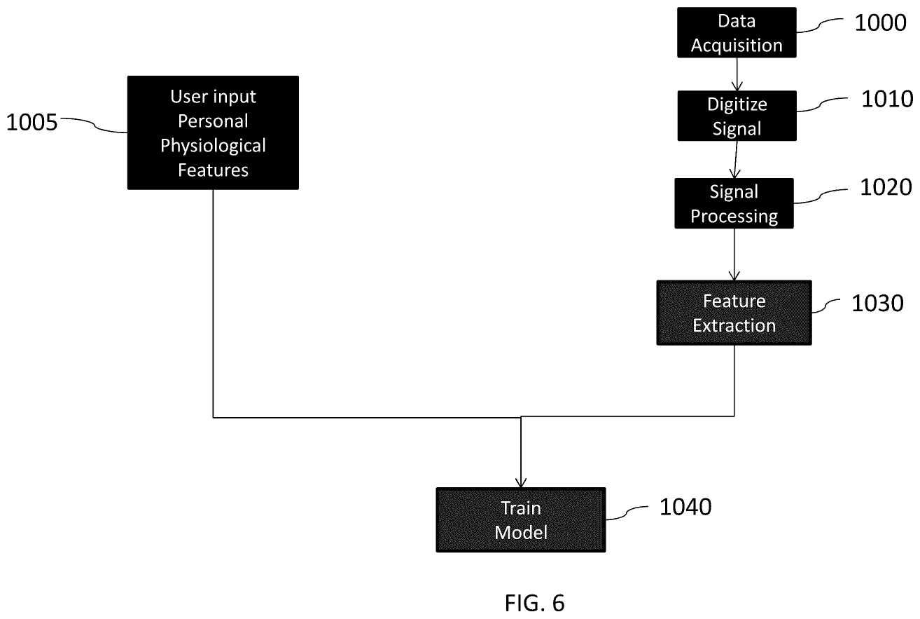

The present invention also provides a method for NIBG neural network prediction based on implicit HbA1c. FIGS. 6, 7 and 8 illustrate the method of the present invention. As shown in FIGS. 6, 7 and 8, the method of the present invention comprises training phase, pretest phase and prediction phase, respectively.

FIG. 6 illustrate the training phase of the method of the present invention. The training phase of the method of the present invention begins with obtaining a set of training data set from training cohort for training the neural network 230 of the present invention. This comprises step 1000 of acquiring cardiovascular signals from each member of a training cohort using signal input 110 as well as step 1005 of inputting each member of the training cohort's physiological features such as BMI, age, waist size, height, weight, etc. . . . In addition, step 1000 involves obtaining reference data set. In one embodiment, the reference data set comprises explicit HbA1c and blood glucose level obtained from each member of the training cohort using conventional finger pricking or drawing of blood method for purposes of input as well as for training the neural network 230 of the present invention. In an embodiment, blood glucose level comprises fasting blood glucose level which is the finger prick glucose level obtained from each person of the training cohort when that person is in a fasting state. In an embodiment, the fasting state is defined as at least about 2 hours, about 3 hours or about 4 hours after the last meal.

In one embodiment, there are more than 100, 500 or 1000 persons in the training cohort. It is preferable that the training cohort comprises a diversity of people of various sexes, ages and physical conditions. In an embodiment, each person of the training cohort may or may not being undergoing any medical treatment. In an embodiment, each person of the training cohort is not undergoing any medical treatment. In another embodiment, each person of the training cohort may or may not be undergoing any medical treatment that can affect his or her cardiovascular system and/or not undergoing any medical treatment for diabetes. In another embodiment, each person of the training cohort is not undergoing any medical treatment that can affect his or her cardiovascular system and/or not undergoing any medical treatment for diabetes.

If the cardiovascular signals are collected in analogue format, the signals are digitized in step 1010 by A/D converter 220. The digitized signal 252 may be stored in database 250 as part of step 1010. Next, the digitized signal 252 is subjected to signal processing in step 1020 by the signal processor 222. In one embodiment, as mentioned above, the signal processing step 1020 may comprise decomposing the signal into high frequency part and low frequency part, such as separating its AC component from the DC component. In another embodiment, the signal processing step 1020 may comprise transforming the signal using transformation methods such as Fourier transformation, wavelet transformation, Hilbert-Huang transformation or any other transformation related to any time-frequency analysis. In an embodiment, the digitized signals are processed into signal windows which are digitized segments of the cardiovascular signal as disclosed above. Results of the signal processing 1020 may be stored in memory 250 as part of step 1020.

Next, in step 1030, the feature extractor 224 extracts extracted features 256 from the digitized signal 252 and/or the processed signal 254 such as pulse morphological features comprising the width of the pulse at 50% height, total pulse area of the minute, average pulse area, the median of the pulse area, time difference from pulse valley to peak or a combination thereof as well as heart rate variance features comprising both low and high-frequency power from fast Fourier transformation (FFT), total power from FFT, percentage of pulse successive interval changes exceeding 20 ms, and/or the standard deviation of successive interval changes or a combination thereof. The extracted features 256 may be stored in memory 250 as part of step 1030.

After extracting the extracted features 256, any embodiments of the NIBG prediction system disclosed may be trained in steps 1040 to estimate blood glucose level using the training data obtained and derived in prior steps. Training methods are known in the art such as discussed in the reference cited in reference 28 which is incorporated in its entirety. In an embodiment, training should result in minimization of summation of loss between reference finger prick BG data and corresponding BG prediction for each person in the training cohort by modifying weights and biases within any embodiments of the neural network 230 of NIBG prediction system of the present invention such as the filters, dense layers etc. In an embodiment, training results in the machine learning system of the present invention learning relationships between HbA1c, blood glucose level and one or more variables wherein the one or more variables comprise various features disclosed herein including personal physiological features, pulse morphological features, and/or heart rate variance features or a combination thereof.

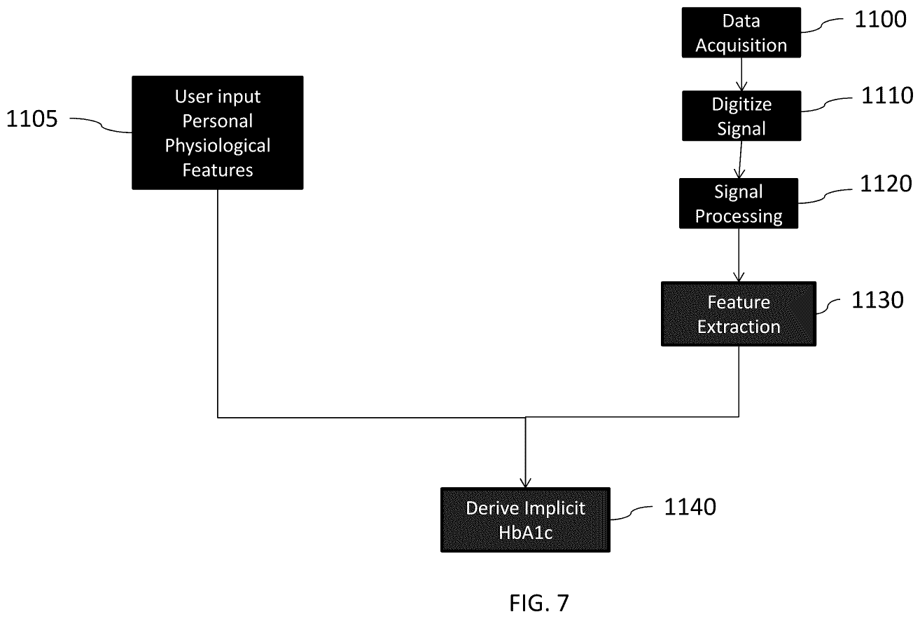

After the training phase, the system of the present invention is configured to relate HbA1c, blood glucose level and the one or more variables such as cardiovascular signal features and/or other features etc. Therefore, an implicit HbA1c may be derived in the next phase which is the pretest phase. An embodiment of the pretest phase is illustrated in FIG. 7. In an embodiment, the implicit HbA1c value may be derived from the system of the present invention using blood glucose level and the one or more variables. As shown in FIG. 7, an embodiment of the pretest phase of the method for estimating blood glucose level of the present invention comprises steps 1100 data acquisition, 1105 user input, 1110 digitize signal, signal processing 1120 and feature extraction 1130. With the exception of step 1100 data acquisition, steps 1105, 1110, 1120 and 1130 are the same steps as the training process steps 1005 user input, 1010 digitize signal, signal processing 1020 and feature extraction 1030, respectively, except that they are performed for one particular user, subject 100, who is interested in NIBG prediction.

Step 1100 data acquisition of the pretest phase differs from step 1000 data acquisition of the training phase in that step 1100 of the pretest phase does not require acquisition of explicit HbA1c. However, step 1100 of the pretest phase does include acquisition of cardiovascular signal and finger prick blood glucose level data form the subject 100. In an embodiment, the finger prick blood glucose level comprises fasting blood glucose level which is the finger prick glucose level obtained from the subject when the subject is in a fasting state. In an embodiment, the fasting state is defined as at least about 2 hours, about 3 hours or about 4 hours after the last meal. In step 1140, the method of the present invention derives implicit HbA1c using finger prick blood glucose level and the one or more variables and the relationships between HbA1c, the finger prick blood glucose level and the one or more variables learned by the neural network during the training phase. In an embodiment, the step 1140 comprises varying the HbA1c as input in the system of the present invention. The system of the present invention would produce a predicted blood glucose level value for each input HbA1c value. In an embodiment, the input HbA1c value may vary from about 4 to 12 of varying increments such as about 0.01 to about 1 such as about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 or 1 including any increment or increment ranges falling within these values. In an embodiment, the implicit HbA1c value is the HbA1c value input into the neural network that produces a predicted blood glucose level closest to the subject's finger prick blood glucose level. In an embodiment, the implicit HbA1c value is the HbA1c value input into the neural network that produces a predicted blood glucose level that is about equal to the subject's finger prick blood glucose level. In an embodiment, the implicit HbA1c value is the HbA1c value input into the neural network that produces a predicted blood glucose level that equals the subject's finger prick blood glucose level. This implicit HbA1c value may then be used for blood glucose prediction using the system of the present invention for the next two to three months.

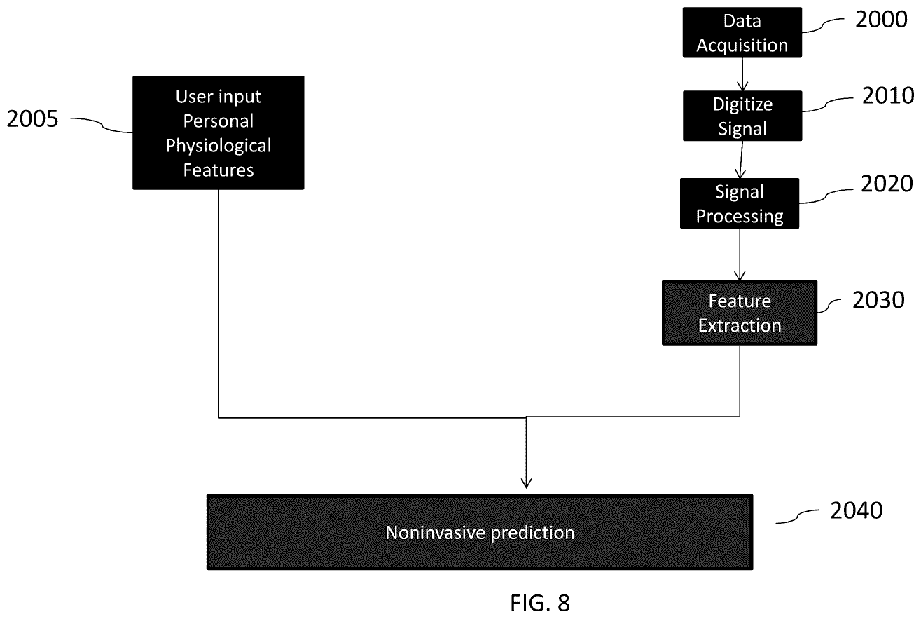

After the training phase and the pretest phase, the NIBG prediction system of the present invention is ready to predict BG level as illustrated in FIG. 8 for subjects who may or may not be undergoing any medical treatments. In an embodiment, the method of the preset invention is used to predict blood glucose level for subjects not undergoing any medical treatments. In an embodiment, the method of the present invention is used to predict blood glucose level for subjects not undergoing any medical treatments that can affect the subject's cardiovascular system and/or subject not undergoing any medical treatments for diabetes. As shown in FIG. 8, the method for estimating blood glucose level comprises steps 2000 data acquisition, 2005 user input, 2010 digitize signal, signal processing 2020 and feature extraction 2030. These are the same steps as the training process steps 1000 data acquisition, 1005 user input, 1010 digitize signal, signal processing 1020 and feature extraction 1030, respectively, except that they are performed for one particular user, subject 100, who is interested in NIBG prediction, and this is the same subject as the subject of the pretest phase so that the implicit HbA1c derived in the pretest phase may be used. In addition, step 2000 differs from step 1000 data acquisition, in that no finger prick blood glucose level is required from the user 100. In addition, step 2000 differs from step 1000 data acquisition in that explicit HbA1c is not obtained from the subject since implicit HbA1c obtain in the pretest phase is used in place of explicit HbA1c for predicting BGL. In step 2040, the trained model is used to perform NIBG prediction using implicit HbA1c derived or calculated during the pretest phase illustrated in FIG. 7.

In an embodiment, in both the pretest and prediction phase, the model is run three or more times for each input set. In the event that the differences in the results exceed a certain threshold, the results are deemed unreliable and should be disregarded. In an embodiment, the threshold is the difference between the largest and the smallest BGL as a percentage of the smallest BGL for each input set.

$$Threshold = 100 \% ⋆ ( {BGL}_{largest} - {BGL}_{smallest} ) / {BGL}_{smallest}$$

In an embodiment, the threshold is ±5%, ±10%, ±15%, ±20%, ±25%, ±30%, ±35%, ±40% or any percentage or percentage ranges falling within these values.

### EXAMPLES

### Materials and Methods

From the original dataset comprising 2632 entries, a subset of 856 entries of data consisting of data from subjects not undergoing drug treatment was meticulously chosen for this study. The dataset is collected from twenty local healthcare centers across Taipei and Taoyuan County with random voluntary participants. During the data collection phase, most of the lower blood glucose subjects were unwilling to participate in the second testing, thus their data were used in model training exclusively. On the other hand, higher blood glucose subjects displayed greater enthusiasm for further testing after a few weeks to monitor changes in their blood glucose levels, as shown in Table 2. Each entry within this subset comprises two consecutive 60 s segments of PPG measurement at a 250 HZ sampling rate collected through transmissive PPG finger clips (infrared, wavelength of 940 nm) on the index finger with the TI AFE4490 Integrated Analog Front End, along with corresponding measurements of blood glucose levels via finger-pricking using the Roche Accu-Chek mobile, HbA1c using the Siemens DCA Vantage Analyzer, and blood pressure using the Omron HEM-7320. The subjects were first asked to sit on the chair in a relaxed position for at least 5 min before the measurements started. During measurement, the blood pressure and finger-prick blood glucose level measurements were taken first, immediately followed by the 60 s long PPG measurement. The collection of these samples received approval from the Institutional Review Board of the Academia Sinica, Taiwan (Application No: AS-IRB01-16081). It is noteworthy that all subjects were comprehensively informed and consented to the collection of the data and their usage.

TABLE 1
Characteristics of the subjects in the training and testing sets
with their mean ± standard deviation.

| | | Interval between Test | BG | HbA1c | Age | BMI |
|---|---|---|---|---|---|---|
| | Dataset | and Pretest | (mg/dL) | (%) | (Years) | (kg/m²) |
| Total | Training | No pretest | 99.9 ± 12.9 | 5.7 ± 0.53 | 57.9 ± 9.7 | 23.4 ± 3.2 |
| 856 | (747 entries) | | | | | |
| entries | Testing | 45 ± 19 days | 154.9 ± 50.8 | 7.7 ± 1.76 | 62.7 ± 3.95 | 28.4 ± 4.3 |
| | (61 pairs) | | | | | |

The 60 s long PPG signals are segmented into windows with a width of 400 data points (equivalent to 1.6 s) based on each pulse valley. A total of 11 features are extracted, encompassing both morphological and heart rate variance (HRV) features. The morphological features include the width of the pulse at half-height, the time taken from pulse valley to pulse peak, the sum of the pulse area of the minute, the average pulse area, and the median of the pulse area. The HRV features include the high, low, and total frequency power from fast Fourier transform (FFT), the percentage of pulse successive interval changes exceeding 20 ms, and the standard deviation of pulse successive interval changes.

In this study, our primary focus is exclusively on subjects who are not undergoing treatment with drugs. This approach serves as a follow-up to our previously proposed method, with the intent of enhancing its effectiveness. Our previous work achieved over 90% accuracy on cohorts not affected by medication with measured HbA1c employed as a feature.

For this work, subjects with multiple entries are deliberately reserved for use as the testing set, while the remaining subjects constitute the training set. The characteristics of both the training and testing sets are concisely outlined in Table 1. To align our approach with practical usage scenarios, a total of 61 pairs, each with a time interval not exceeding 90 days, are utilized for testing. Evaluating performance beyond the three-month validity of HbA1c would be both impractical and devoid of meaningful insights. Thus, the data pairs with intervals exceeding 90 days were further excluded from the testing. Within the testing set, each subject's multiple rounds of measurements are meticulously paired together in a sequential manner to establish the testing data structure. Each pair is composed of a pretest and a test measurement, collected from different measurement rounds, thereby forming varying time intervals. The valid time interval between the pretest and test spans from 11 to 90 days. Notably, none of the measurements belonging to subjects designated for the testing set are used in the training set. This meticulous separation ensures the establishment of the strictest testing condition, where the model has not yet been influenced by any prior measurements of the intended test subjects.

The methodology proposed in this study revolves around the utilization of a pretest round to derive an implicit HbA1c value, subsequently enhancing the accuracy of blood glucose level (BGL) predictions during the testing round.

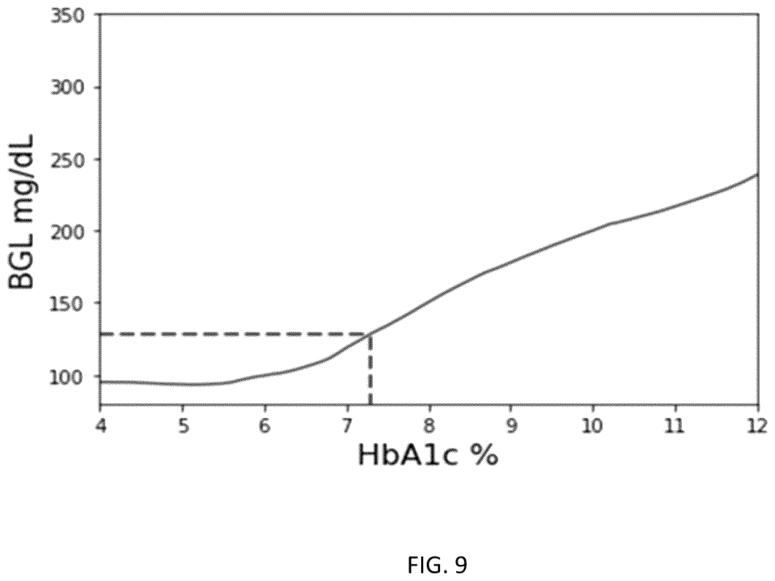

The workflow of this method is depicted in FIGS. 6, 7 and 8. Firstly, a set of three BGL prediction models are trained employing PPG signals, PPG-extracted features, and HbA1c as inputs. These models use identical structures and will be the only prediction models used throughout the method. The three models' purpose is to validate outcomes by cross-referencing with each other. During the pretest phase, a PPG measurement with a corresponding finger-prick BGL reading is taken and inversely applied to the prediction model. The pretest PPG data are then input into the model with varying HbA1c values ranging from 4 to 12 in increments of 0.1. Consequently, a series of BGL predictions is generated, as exemplified in FIG. 9. Among these predictions, the value closest to the measured finger-prick BGL reading is identified, and the corresponding HbA1c value is selected as the implicit HbA1c. This process is repeated independently for each of the three models. The disparities among the outcomes from the three models are assessed to ensure they fall within an acceptable threshold for consistency. Among the three results, the median value is chosen to serve as the designated implicit HbA1c.

During the testing phase, only the PPG measurement is collected and then joined with the pretest-determined implicit HbA1c as input for the model to generate the BGL prediction. Once more, both the PPG measurement and implicit HbA1c are independently input into the three models, and the differences among the prediction outcomes are assessed to ensure their consistency. In the event that the differences among models with identical structures and the same input data exceed a certain threshold the results are deemed unreliable and should be disregarded. This process represents a straightforward and simple approach, leveraging preexisting models to obtain an alternative HbA1c value that enhances the accuracy of BGL predictions.

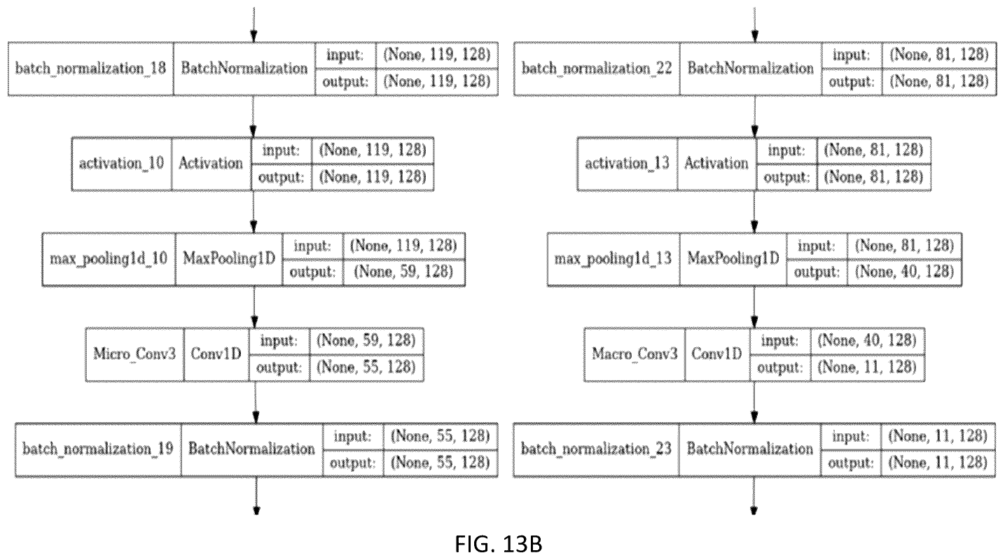

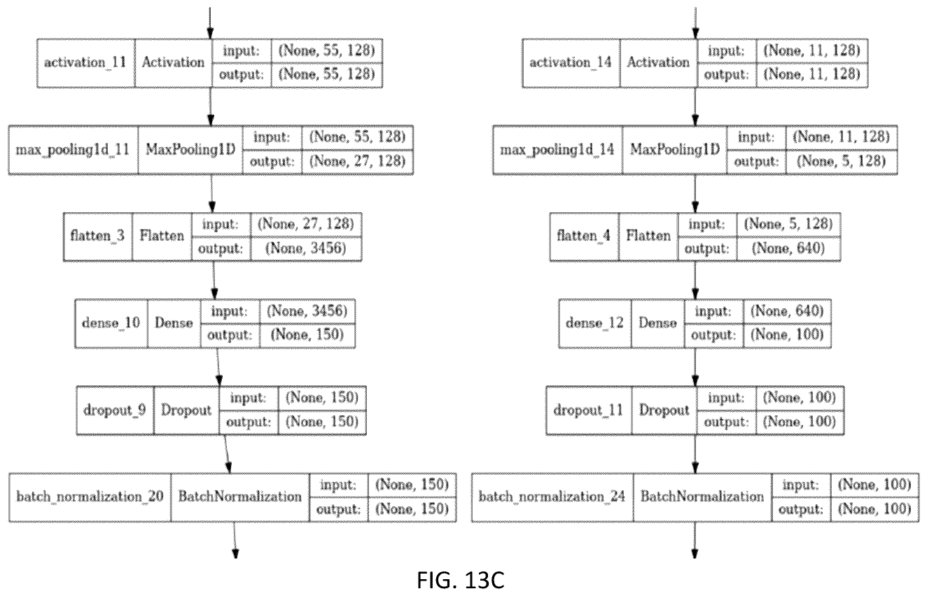

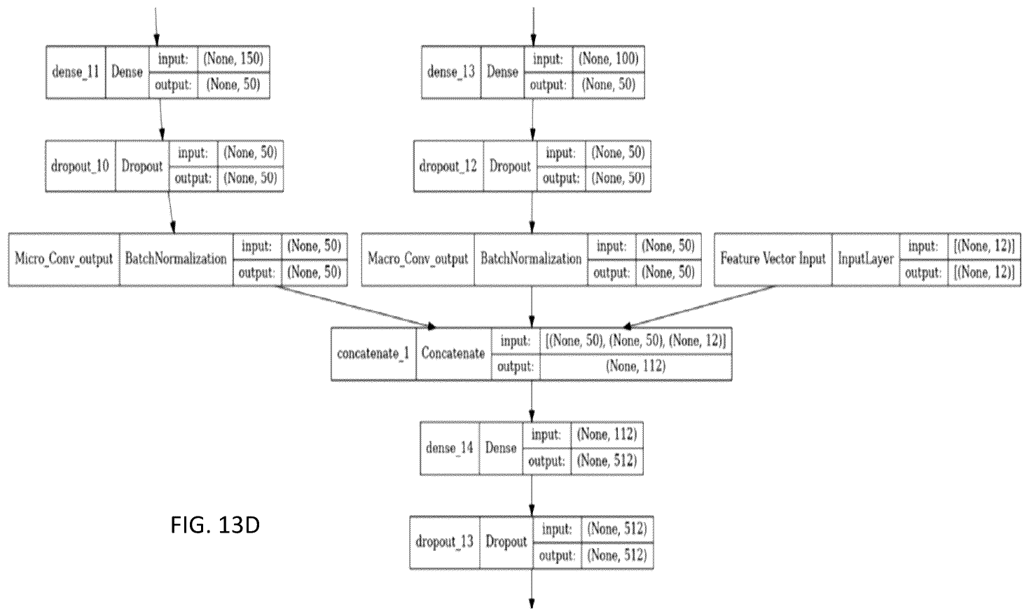

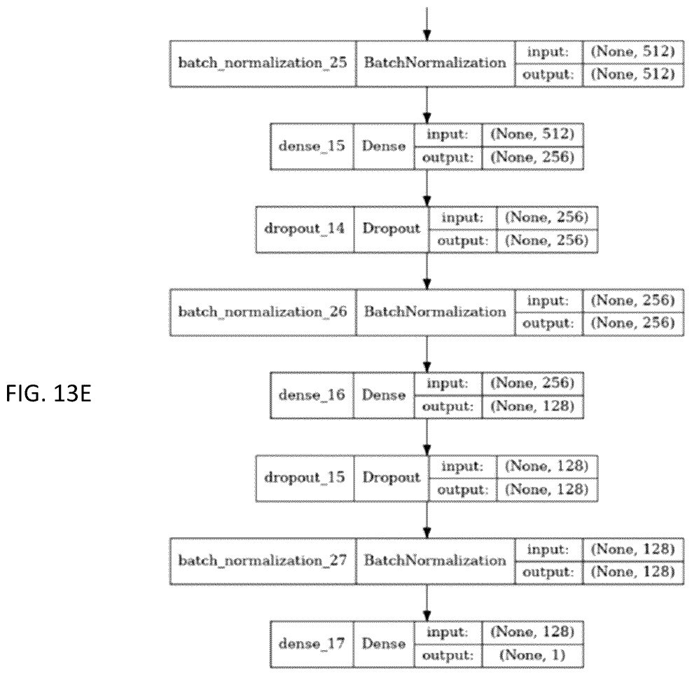

In this study, we utilized Python version 3.9.13 and Keras version 2.7 with tensorFlow version 2.7 as the backend for model building. The BGL prediction model utilized in this study used an identical structure to our prior HbA1c-based method, thus facilitating objective comparisons. The detailed model structure design with every layer can be found in FIG. 13. The model comprises two parallel one-dimensional convolutional neural network (CNN) blocks as illustrated in FIG. 3C, each featuring different filter lengths to encompass both micro and macro perspectives of the input signal window. The CNN outputs are subsequently concatenated with a manually extracted feature vector, which includes the HbA1c measurement. This combined information is then passed through several fully connected layers to generate the BGL prediction output. A comprehensive depiction and in-depth design of the model's structure can be found in our earlier work, where the model achieved a prediction accuracy of over 94% within Clarke's error grid (CEG) zone A for subjects not influenced by any form of medication.

### Results

In this study, Clarke's error grid (CEG) analysis is used as the main performance indicator, as the ISO 15197:2013 (International Organization for Standardization) recommendation requires personal use glucose meters to have 99% of the measurement within CEG's zones A and B [20]. Clarke's error grid analysis is a graphical method used to evaluate the accuracy of blood glucose meters developed by David Clarke in 1987 as a way to assess the clinical significance of errors in glucose measurements. CEG consists of five zones from A to E, each reflecting different clinical significance [21]. Zone A represents an accurate prediction where any differences between the prediction and reference values are considered negligible. Zone B reflects a prediction with a clinically acceptable error which could lead to unnecessary treatment but does not have a significant impact. As for Zones C to E, they represent different degrees of danger to users; if the result is used for clinical purposes, it could lead to severe harm or even death.

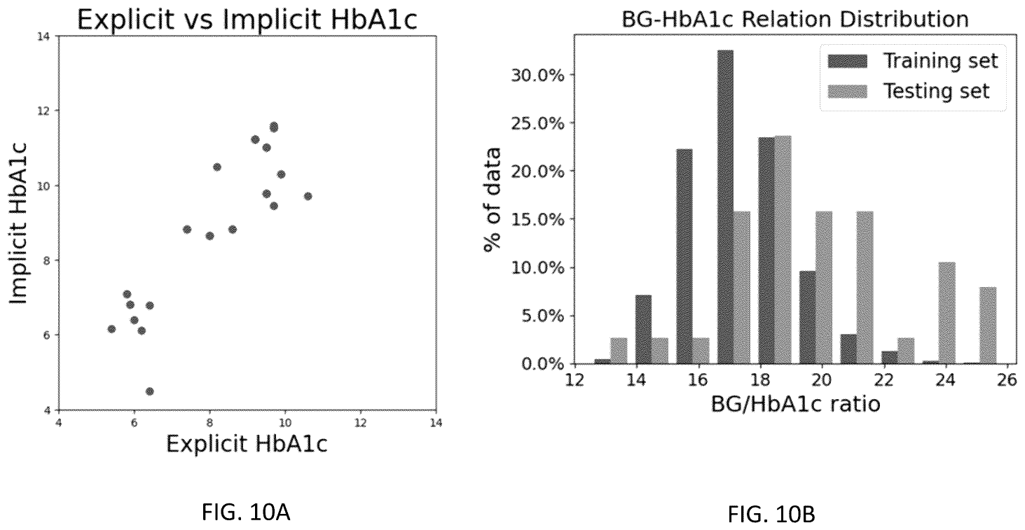

In FIG. 10A, we presented the difference between implicit HbA1c and its corresponding measured reference HbA1c. The graph exhibits a rough alignment with the diagonal line, while implicit HbA1c values appear systematically higher than their corresponding explicit HbA1c values. Implicit HbA1c reflects the HbA1c value that the model anticipates given a specific blood glucose level. This phenomenon can be attributed to the fundamental difference between the training and testing sets we used.

As a result, the training set we used (subjects without multiple entries of measurement) is predominantly composed of subjects with lower blood glucose level. In contrast, the testing set is predominantly composed of individuals with prediabetes and diabetes. Due to the methodology employed, the testing set required the test subjects to have two measurements (pretest and test), but the training set did not require a pretest for model training. This makes it impossible to mix the data between training and testing sets to achieve a more balanced distribution between the two sets. Table 1 provides a glimpse of the notable differences in average HbA1c and blood glucose levels between the training and testing datasets. From FIG. 10B we can see the clear difference in the distribution of the BG-HbA1c relation between our training and testing datasets. Consequently, as we proceed with the process of calculating implicit HbA1c, the elevated fasting blood glucose levels observed within the testing subjects contribute to higher implicit HbA1c values. The systematic deviation between the explicit and implicit HbA1c values does not reflect the error; instead, it shows the amount of correction items adjusted by the methodology to bridge the population for an accurate BGL estimation.

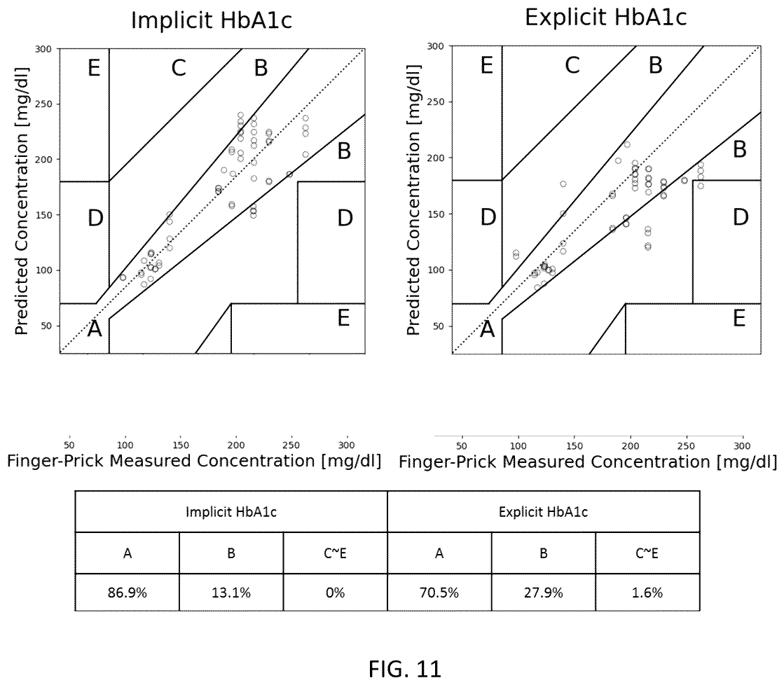

For comparison, a set of predictions was also conducted using explicit HbA1c. This comparative analysis was carried out using the same testing dataset. The overall prediction performances by CEG's zone ratios are documented and summarized in FIG. 11. In the figure, we can see the difference in prediction ability between using the newly proposed implicit HbA1c and the previously used explicit HbA1c. Overall, while using implicit HbA1c, the model not only alleviates the inconvenience associated with HbA1c measurement, but also leads to a substantial 16% improvement in prediction accuracy. This outcome is an indication that implicit HbA1c can be more effective than measured HbA1c. This intriguing phenomenon is hypothesized to stem from the implicit HbA1c calculation process which also introduced a degree of adjustment of personal deviation.

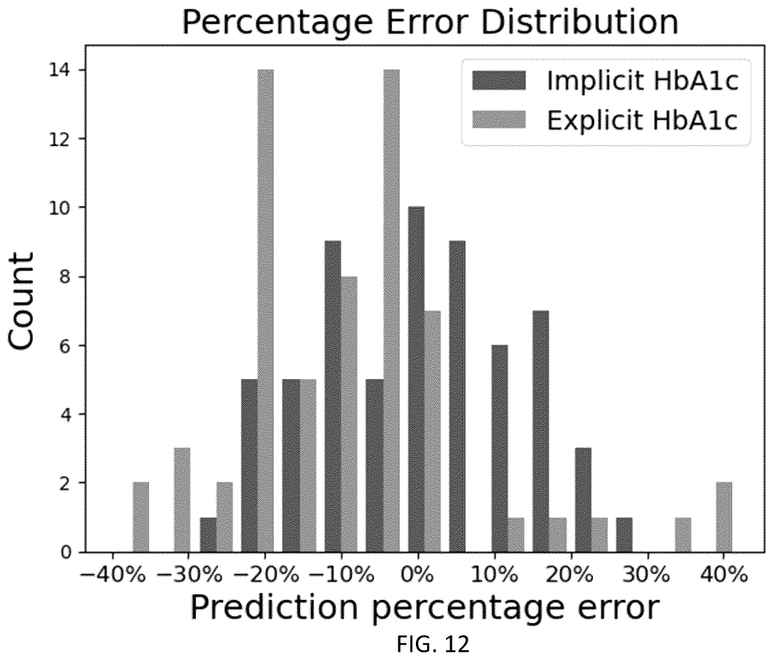

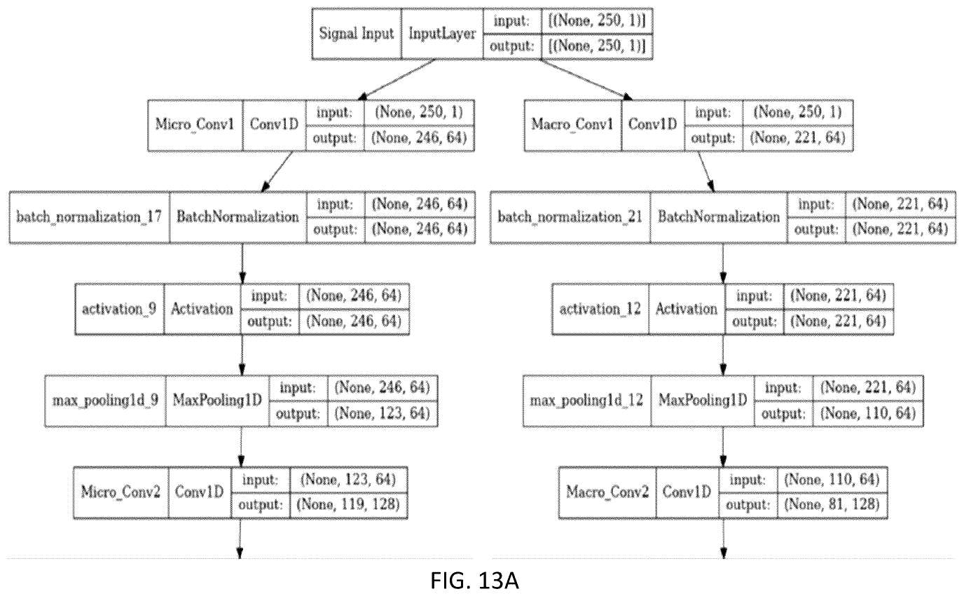

The distribution of the prediction percentage error of using the implicit HbA1c and explicit HbA1c methods is presented in FIG. 12. In the figure, we can see the prediction percentage errors from the implicit HbA1c method exhibit a normal distribution, while those from the explicit HbA1c method displayed a left-skewed distribution with systemically lower prediction. To ascertain the significance of the difference in model performance, a nonparametric Wilcoxon signed-rank test was conducted based on the percentage errors. The statistical result revealed that there are significant differences in prediction accuracy between the two methods, with a p-value of 2.75×10⁻⁷, much smaller than the significance level of 0.05.

### Discussion

The accurate estimation of blood glucose levels (BGL) from non-medicated subjects can be achieved through a machine learning (ML) model that utilizes both photoplethysmography and HbA1c input, as we have previously demonstrated in our work published in Sensors([18]. In that study, the HbA1c measurements used were taken simultaneously under the assumption that they could represent any recently measured HbA1c value with limited degradation in performance, given that HbA1c reflects a three-month average of blood glucose concentration. The less-than-ideal performance on the prediction results when using explicit HbA1c in this study was expected due to the previously mentioned disparities between the training and testing sets, as well as the increased time interval when compared to our prior work. Despite these increased challenges, the implicit HbA1c method effectively generates accurate predictions. This highlights the efficacy of implicit HbA1c in covering correction items from personal deviations.

A machine learning model for BGL estimation can generally be represented as Equation (1). Here, the function ML ( ) symbolizes the machine learning model, while F1 through Fn correspond to the diverse set of features that collectively contribute to achieving an accurate prediction of the blood glucose level.

$$BGL = ML ( F 1 , F 2 , F 3 … Fn ) \qquad ( 1 )$$

While different methods may employ different features, our prior work demonstrated that BGL can be accurately estimated by a machine learning model with PPG and HbA1c input, albeit under certain conditions. This leads us to modify Equation (1) into Equation (2a):

$$BGL = ML ( PPG , HbA 1 c ) \qquad ( 2 a )$$

However, it is important to acknowledge the intricate interplay of variables such as medication, individual differences, lifestyle variations, and more, which may not have been fully accounted for. This realization prompts us to introduce the correction item ΣCi into the equation. For subjects not undergoing treatment with drugs, the effects of ΣCi may not be significant enough to seriously hinder the prediction performance, but it is undeniable that these effects still exist. Consequently, we further revise the equation into Equation (2b).

$$BGL = ML ( PPG , HbA 1 c , \sum Ci ) \qquad ( 2 b )$$

These personal difference effects were dealt with by using a personalized deduction learning model that required multiple measurements from the user in our previous work [22]. Other works sought to account for these deviations by utilizing numerous personal profiles [23]. In this study, we leverage the concept of implicit HbA1c to achieve a similar effect.

Implicit HbA1c is determined by substituting HbA1c and BLG in Equation (2). It is like solving a multi-variate polynomial function with only one unknown variable. To solve for the unknown HbA1c value, the model is provided with a range of HbA1c inputs, generating a series of predictions. By cross-reference these predictions with the known BGL value, we can determine which corresponding HbA1c produces the most accurate estimation. This process not only yielded an HbA1c estimate, but it also accounted for the aforementioned correction items ΣCi. In other words, implicit HbA1c is the HbA1c value that has been adjusted to accommodate an individual's specific correction items. Thus, this refinement further transforms Equation (2b) into Equation (3)

$$BGL = ML ( PPG , HbA 1 {c}_{imp} ) \qquad ( 3 )$$

HbA1c reflects an average BGL, and its correlation with fasting BGL is influenced by individual lifestyle, such as constant high BGL during the day and multiple meals. Consequently, the relationship between each individual's HbA1c and fasting blood glucose follows a unique curve. For instance, individuals with prediabetes may still have a pancreas capable of producing a sufficient amount of insulin to maintain normal fasting BGL, but their daily BGL may fluctuate in a big range depending on diet and lifestyle. We anticipation that the proposed method will demonstrate effectiveness across various demographics, including different races, ages, and genders, as it effectively compensates for personal deviations arising from miscellaneous correction factors.

The self-monitoring of blood glucose (SMBG) serves as an indicator of daily sugar control status in modern diabetes treatment, and its importance might be introducing behavior changes, improving glycemic control, and optimizing therapy [24]. Intensive insulin therapy is usually accompanied by daily SMBG and has proved to reduce the end-organ damage in patients with insulin-dependent diabetes mellitus [25]. There is also evidence suggesting the benefit in pre-diabetic patients or those

under oral anti-diabetic drugs [26]. Some diabetes guidelines suggest SMBG use not only while fasting but also in the post-prandial stage, because the post-prandial glucose excursion, measured by the delta change in fasting and post-prandial sugar, has been demonstrated to correlate with cardiovascular risk [24]. Hence, the structured SMBG protocol by performing glucose tests before and after a meal in pairs has been evaluated in clinical trials and improves glycemic control [27]. Our implicit HbA1c method may increase the frequency of sugar monitoring compared to the guideline-suggested 2˜3 times of SMBG per week in non-insulin-treated T2DM; the usage of this novel non-invasive glucose monitor technology might help diabetologists to optimize diabetic therapy in the future. However, our original dataset was collected in a fasting population; thus, the reliability of post-prandial sugar use remains uncertain. In addition, our prediction model in the insulin-treated population, whose SMBG assessments are in most demand, is less powerful than those not undergoing drug treatment. A further improvement of our algorithm and studies including a broader spectrum of diabetic populations are mandatory.

### Conclusions

The significance of HbA1c as a valuable feature for non-invasive blood glucose prediction is widely acknowledged, although the inconvenience of acquiring HbA1c measurements remains. The HbA1c measurements are generally only available in hospitals or specialized clinics. To tackle this issue, this study introduced an innovative approach known as implicit HbA1c value which derives an alternative HbA1c that only requires a single finger-prick blood glucose measurement and can be easily conducted at home by the users. Implicit HbA1c was introduced as a solution to enable accurate glucose predictions without the need for direct HbA1c measurements with specialized equipment, and it also demonstrated the ability to further improve the prediction performance. The implicit HbA1c method achieved 87% of the prediction results within CEG's zone A, and the remaining 13% close to the zone A boundary. The implicit HbA1c approach not only exhibited a remarkable 16% improvement over the measured HbA1c method by covering personal correction items automatically, but also demonstrated an extended prediction validity period with testing data from 11 up to 90 days. The nonparametric Wilcoxon paired test conducted on the percentage error suggests a statistically significant difference between their performances with a p-value of 2.75×10⁻⁷.

### REFERENCES (EACH INCORPORATED IN ITS ENTIRETY)

1. World Health Organization Diabetes. [(accessed on 28 Aug. 2023)]; Available online: https://www.who.int/news-room/fact-sheets/detail/diabetes
2. Centers for Disease Control and Prevention National and State Diabetes Trends. [(accessed on 28 Aug. 2023)]; Available online: https://www.cdc.gov/diabetes/library/reports/reportcard/national-state-diabetes-trends. html
3. International Diabetes Federation Diabetes around the World in 2021. [(accessed on 28 Aug. 2023)]. Available online: https://diabetesatlas.org/4. UK Prospective Diabetes Study (UKPDS) Group Intensive blood-glucose control with sulphonylureas or insulin compared with conventional treatment and risk of complications in patients with type 2 diabetes (UKPDS 33) Lancet. 1998;352:837-853. doi: 10.1016/S0140-6736 (98) 07019-6. [PubMed] [CrossRef] [Google Scholar]
5. Aihara M., Kubota N., Minami T., Shirakawa R., Sakurai Y., Hayashi T., Iwamoto M., Takamoto I., Kubota T., Suzuki R., et al. Association between tear and blood glucose concentrations: Random intercept model adjusted with confounders in tear samples negative for occult blood. J. Diabetes Investig. 2021; 12:266-276. doi: 10.1111/jdi.13344. [PMC free article] [PubMed] [CrossRef] [Google Scholar]
6. Zafar H., Channa A., Jeoti V., Stojanovic G. M. Comprehensive Review on Wearable Sweat-Glucose Sensors for Continuous Glucose Monitoring. Sensors. 2022;22:638. doi: 10.3390/s22020638. [PMC free article] [PubMed] [CrossRef] [Google Scholar]
7. Zhang W., Du Y., Wang M. L. Noninvasive glucose monitoring using saliva nano-biosensor. Sens. Bio-Sens. Res. 2015;4:23-29. doi: 10.1016/j.sbsr.2015.02.002. [CrossRef] [Google Scholar]
8. Monte-Moreno E. Non-invasive estimate of blood glucose and blood pressure from a photoplethysmograph by means of machine learning techniques. Artif. Intell. Med. 2011;53:127-138. doi: 10.1016/j.artmed.2011.05.001. [PubMed] [CrossRef] [Google Scholar]
9. Rachim V. P., Chung W.-Y. Wearable-band type visible-near infrared optical biosensor for non-invasive blood glucose monitoring. Sens. Actuators B Chem. 2019;286:173-180. doi: 10.1016/j.snb.2019.01.121. [CrossRef] [Google Scholar]
10. Yadav J., Rani A., Singh V., Murari B. M. Prospects and limitations of non-invasive blood glucose monitoring using near-infrared spectroscopy. Biomed. Signal Process. Control. 2015;18:214-227. doi: 10.1016/j.bspc.2015.01.005. [CrossRef] [Google Scholar]
11. Pedro B. G., Marcondes D. W. C., Bertemes-Filho P. Analytical Model for Blood Glucose Detection Using Electrical Impedance Spectroscopy. Sensors. 2020;20:6928. doi: 10.3390/s20236928. [PMC free article] [PubMed] [CrossRef] [Google Scholar]
12. Alian A. A., Shelley K. H. Photoplethysmography. Best. Pract. Res. Clin. Anaesthesiol. 2014;28:395-406. doi: 10.1016/j.bpa.2014.08.006. [PubMed] [CrossRef] [Google Scholar]
13. den Brinker A. C., Wang W. Chapter 3-Model-based camera-PPG: Pulse-rate monitoring in fitness. In: Wang W., Wang X., editors. Contactless Vital Signs Monitoring. Academic Press; Cambridge, MA, USA: 2022. pp. 51-78. [Google Scholar]
14. Hina A., Saadeh W. Noninvasive Blood Glucose Monitoring Systems Using Near-Infrared Technology-A Review. Sensors. 2022;22:4855. doi: 10.3390/s22134855. [PMC free article] [PubMed] [CrossRef] [Google Scholar]
15. Benichou T., Pereira B., Mermillod M., Tauveron I., Pfabigan D., Maqdasy S., Dutheil F. Heart rate variability in type 2 diabetes mellitus: A systematic review and meta-analysis. PLOS ONE. 2018; 13: e0195166. doi: 10.1371/journal.pone.0195166. [PMC free article] [PubMed] [CrossRef] [Google Scholar]
16. Paneni F., Beckman J. A., Creager M. A., Cosentino F. Diabetes and vascular disease: Pathophysiology, clinical consequences, and medical therapy: Part I. Eur. Heart J. 2013; 34:2436-2443. doi: 10.1093/eurheartj/eht149. [PMC free article] [PubMed] [CrossRef] [Google Scholar]
17. Hina A., Saadeh W. A Noninvasive Glucose Monitoring SoC Based on Single Wavelength Photoplethysmography. IEEE Trans. Biomed. Circuits Syst. 2020; 14:504-515. doi: 10.1109/TBCAS.2020.2979514. [PubMed] [CrossRef] [Google Scholar]
18. Chu J., Yang W. T., Lu W. R., Chang Y. T., Hsieh T. H., Yang F. L. 90% Accuracy for Photoplethysmography-Based Non-Invasive Blood Glucose Prediction by Deep Learning with Cohort Arrangement and Quarterly Measured HbA1c. Sensors. 2021;21:7815. doi: 10.3390/s21237815. [PMC free article] [PubMed] [CrossRef] [Google Scholar]
19. World Health Organization. Use of Glycated Haemoglobin (HbA1c) in the Diagnosis of Diabetes Mellitus: Abbreviated Report of a WHO Consultation. World Health Organization CTI-WHO Guidelines Approved by the Guidelines Review Committee; Geneva, Switzerland: 2011. [PubMed] [Google Scholar]
20. International Organization for Standardization. In Vitro Diagnostic Test Systems-Requirements for Blood-Glucose Monitoring Systems for Self-Testing in Managing Diabetes Mellitus. ISO; Geneva, Switzerland: 2013. [Google Scholar]
21. Clarke W. L., Cox D., Gonder-Frederick L. A., Carter W., Pohl S. L. Evaluating clinical accuracy of systems for self-monitoring of blood glucose. Diabetes Care. 1987;10:622-628. doi: 10.2337/diacare. 10.5.622. [PubMed] [CrossRef] [Google Scholar]
22. Lu W. R., Yang W. T., Chu J., Hsieh T. H., Yang F. L. Deduction learning for precise noninvasive measurements of blood glucose with a dozen rounds of data for model training. Sci. Rep. 2022; 12:6506. doi: 10.1038/s41598-022-10360-3. [PMC free article] [PubMed] [CrossRef] [Google Scholar]
23. Hettiarachchi C., Chitraranjan C. A Machine Learning Approach to Predict Diabetes Using Short Recorded Photoplethysmography and Physiological Characteristics; Proceedings of the Artificial Intelligence in Medicine; Poznan, Poland. 26-29 Jun. 2019; pp. 322-327. [Google Scholar]
24. International Diabetes Federation Clinical Guidelines Task Force Guideline on Self-Monitoring of Blood Glucose in Non-Insulin Treated Type 2 Diabetes. Int. Diabetes Fed. 2009; 17:4. [Google Scholar]
25. Nathan D. M., Genuth S., Lachin J., Cleary P., Crofford O., Davis M., Rand L., Siebert C. The effect of intensive treatment of diabetes on the development and progression of long-term complications in insulin-dependent diabetes mellitus. N. Engl. J. Med. 1993;329:977-986. doi: 10.1056/NEJM199309303291401. [PubMed] [CrossRef] [Google Scholar]
26. Sia H. K., Kor C. T., Tu S. T., Liao P. Y., Wang J. Y. Self-monitoring of blood glucose in association with glycemic control in newly diagnosed non-insulin-treated diabetes patients: A retrospective cohort study. Sci. Rep. 2021; 11:1176. doi: 10.1038/s41598-021-81024-x. [PMC free article] [PubMed] [CrossRef] [Google Scholar]
27. Di Molfetta S., Bosi E., Ceriello A., Cucinotta D., Tiengo A., Scavini M., Piccolo C., Bonizzoni E., Acmet E., Giorgino F., et al. Structured self-monitoring of blood glucose is associated with more appropriate therapeutic interventions than unstructured self-monitoring: A novel analysis of data from the PRISMA trial. Diabetes Res. Clin. Pract. 2021; 181:109070. doi: 10.1016/j.diabres.2021.109070. [PubMed] [CrossRef] [Google Scholar]
28. Sarkar, K., et al. Design and Implementation of a Noninvasive Blood Glucose Monitoring Device. in 2018 21st International Conference of Computer and Information Technology (ICCIT). 2018. Dhaka, Bangladesh: IEEE.

What is claimed is:

1. A non-invasive blood glucose (NIBG) prediction system comprising
a cardiovascular signal input configured to input one or more cardiovascular signals of a subject into the system,
a processor comprising a signal processor wherein the signal processor is configured to perform signal processing on the one or more cardiovascular signals to extract one or more cardiovascular signal features and the processor further comprising a neural network (NN) wherein the NN is configured to predict a subject's blood glucose level based on implicit HbA1c;
wherein the NN comprises one or more variables;
wherein the one or more variables comprises the one or more cardiovascular signal features;
wherein the NN predicts blood glucose level of the subject using the one or more variables and the implicit HbA1c; and
wherein the implicit HbA1c is HbA1c derived from the NN in a pretest phase based on finger prick blood glucose level of the subject and the one or more variables wherein the pretest phase occurs prior to blood glucose level prediction phase but after training phase of the NN.

2. The system of claim 1, wherein the NN comprises one or more relationships between HbA1c, blood glucose level and the one or more variables wherein the relationships are learned by the NN during the training phase.

3. The system of claim 2, wherein the implicit HbA1c is HbA1c derived from the NN using the finger prick blood glucose level, the one or more variables and the one or more relationships between HbA1c, the blood glucose level and the one or more variables.

4. The system of claim 1 wherein the cardiovascular signal comprises photoplethysmography (PPG) signals and the cardiovascular signal features comprises PPG features.

5. The system of claim 1, wherein the implicit HbA1c is the HbA1c value input into the NN that renders the NN to predict a blood glucose level closest to the finger prick blood glucose level of the subject out of all the HbA1c values input into the NN during the pretest phase.

6. The system of claim 1, wherein the implicit HbA1c is the HbA1c value input into the NN that renders the NN to predict a blood glucose level that is about equal to the finger prick blood glucose level of the subject during the pretest phase.

7. The system of claim 1, wherein the implicit HbA1c does not comprise explicit HbA1c wherein the explicit HbA1c is HbA1c measured from the subject using HbA1c medical diagnostic devices.

8. The system of claim 1, wherein the NN is trained in the training phase of the NN using training data obtained from a training cohort not undergoing any medical treatment that can affect the cardiovascular system of the training cohort and/or undergoing any diabetes drugs or diabetes treatments.

9. The system of claim 1, wherein the subject is not undergoing any medical treatment that can affect the cardiovascular system of the subject or any or undergoing any diabetes drugs or diabetes treatments.

10. The system of claim 1, wherein the subject is not taking any drugs or undergoing any medical treatments or procedures for treating high blood pressure and high blood pressure prevention, stroke and stroke prevention drugs and heart attack and heart attack prevention drugs and/or diabetes drugs or diabetes treatments or a combination thereof.

11. The system of claim 1, wherein the one or more variables comprises personal physiological features, pulse morphological features, and/or heart rate variance features or a combination thereof wherein the pulse morphological features and heart rate variance features are features extracted by the processor from the one or more cardiovascular signals.

12. The system of claim 11, wherein the personal physiological features comprise age, waist circumference, body mass index, systolic blood pressure, and/or diastolic blood pressure or a combination thereof.

13. The system of claim 11, wherein the pulse morphological features comprise the width of the pulse at 50% height, total pulse area of the minute, average pulse area, the median of the pulse area, and/or time difference from pulse valley to peak or a combination thereof.

14. The system of claim 11, wherein the heart rate variance features comprise low-frequency power from fast Fourier transformation (FFT), high-frequency power from FFT, total power from FFT, percentage of pulse successive interval changes exceeding 20 ms, and/or standard deviation of successive interval changes or a combination thereof.

15. The system of claim 1, wherein the NN comprises a convolution neural network (CNN).

16. The system of claim 15, wherein the CNN comprises one or more convolution layers wherein each convolution layer is configured to perform convolution operation on the one or more inputs or output from another convolution layer.

17. The system of claim 16 wherein the CNN comprises two or more CNN modules in parallel.

18. The system of claim 17 wherein each CNN module comprises filters of different lengths compared to each of the filters of other CNN modules.

19. The system of claim 18, wherein the CNN comprise two CNN modules and wherein filter length for one CNN module is ¼ to ¾ the length of the filter length of the other CNN module.

20. They system of claim 17, wherein the CNN further comprises a merging block configured to merge and analyze output of each of the CNN module.

21. The system of claim 1, wherein the processor creates a signal window as input to the NN by digitizing a segment of the one or more cardiovascular signals.

22. The system of claim 1, wherein the signal input comprises a cardiovascular signal reader.

23. A method for non-invasive blood glucose (NIBG) prediction based on implicit HbA1c comprising the steps of:
training a neural network (NN) of the processor during training phase using explicit HbA1c, finger prick blood glucose level, and one or more variables obtained from each member of a training cohort so that the NN learns one or more relationships between the HbA1c, the blood glucose level, the one or more variables wherein the one or more variables comprise one or more cardiovascular signal features;
inputting one or more cardiovascular signals of a subject to a processor;
processing the one or more cardiovascular signals of the subject using the processor to obtain one or more cardiovascular signal features of the subject;
obtaining explicit HbA1c and finger prick blood glucose level from the subject wherein explicit HbA1c is HbA1c measured from the subject using medical diagnostic devices and finger prick blood glucose level is blood glucose level measured from the subject using medical diagnostic devices;
deriving an implicit HbA1c of the subject from the trained NN during pretest phase of the NN based on the finger prick blood glucose level of the subject, one or more variables of the subject and the one or more relationships between HbA1c, blood glucose level, one or more variables learned by the NN during the training phase; and
predicting blood glucose level of the subject using the trained NN based on the implicit HbA1c of the subject.

24. The method of claim 23, wherein the cardiovascular signal comprises photoplethysmography (PPG) signals and the cardiovascular signal features comprises PPG features.

25. The method of claim 23, wherein the implicit HbA1c is the HbA1c value input into the NN that renders the NN to predict a blood glucose level closest to finger prick blood glucose level of the subject out of all the HbA1c values input into the NN during the pretest phase.

26. The method of claim 23, wherein the implicit HbA1c is the HbA1c value input into the NN that renders the NN to predict a blood glucose level that is about the finger prick blood glucose level of the subject.

27. The system of claim 23, wherein the implicit HbA1c is not explicit HbA1c wherein the explicit HbA1c is HbA1c measured directly from the subject using diagnostic devices.

28. The method of claim 23, wherein the subject and each member of the training cohort are not undergoing any medical treatments that can affect the cardiovascular system and/or undergoing any diabetes drugs or diabetes treatments.

29. The method of claim 23, wherein the subject and the training cohort are not taking any drugs or undergoing any medical treatments or procedures for treating high blood pressure and high blood pressure prevention, stroke and stroke prevention drugs and heart attack and heart attack prevention drugs and/or diabetes drugs or treatments such as insulin injections or a combination thereof.

30. The method of claim 23, wherein the one or more variables further comprises personal physiological features.

31. The method of claim 23, wherein the cardiovascular features comprise pulse morphological features and heart rate variance features.

32. The method of claim 30, wherein the personal physiological features comprise age, waist circumference, body mass index, systolic blood pressure, diastolic blood pressure or a combination thereof.

33. The method of claim 31, wherein the pulse morphological features comprise the width of the pulse at 50% height, total pulse area of the minute, average pulse area, the median of the pulse area, and/or time difference from pulse valley to peak or a combination thereof.

34. The method of claim 31, wherein the heart rate variance features comprise low-frequency power from fast Fourier transformation (FFT), high-frequency power from FFT, total power from FFT, percentage of pulse successive interval changes exceeding 20 ms, and/or standard deviation of successive interval changes or a combination thereof.

35. The method of claim 23, wherein the NN comprises a convolution neural network (CNN).

36. The method of claim 35, wherein the CNN comprises one or more convolution layers wherein each convolution layer is configured to perform convolution operation on the one or more inputs or output from another convolution layer.

37. The method of claim 36, wherein the CNN comprises two or more CNN modules in parallel.

38. The method of claim 37, wherein each CNN module comprises filters of different lengths compared to each of the filters of the other CNN modules.

39. The method of claim 38, wherein the CNN comprise two CNN modules and wherein filter length for one CNN module is ¼ to ¾ the length of the filter length of the other CNN module.

40. They method of claim 37, wherein the CNN further comprises a merging block configured to merge and analyze output of each CNN module.

41. The method of claim 23, wherein the processing step comprises creating a signal window as input to the NN by digitizing a segment of one of the one or more PPG signal.
